(19) United States
(12) Patent Application Publication
(10) Pub. No.: US 2024/0350689 A1
(43) Pub. Date: Oct. 24, 2024

(54) ACTIVE OXYGEN SUPPLY DEVICE, DEVICE FOR PERFORMING TREATMENT WITH ACTIVE OXYGEN, AND METHOD FOR PERFORMING TREATMENT WITH ACTIVE OXYGEN

(71) Applicant: CANON KABUSHIKI KAISHA, Tokyo (JP)

(72) Inventors: TAKUMI FURUKAWA, Shizuoka (JP); MASAKI OZAWA, Shizuoka (JP); KAZUHIRO YAMAUCHI, Shizuoka (JP); KENJI TAKASHIMA, Kanagawa (JP); MOTOTERU GOTO, Shizuoka (JP); SHOTA KANEKO, Tokyo (JP)

(21) Appl. No.: 18/751,455

(22) Filed: Jun. 24, 2024

Related U.S. Application Data
(63) Continuation of application No. PCT/JP2022/048035, filed on Dec. 26, 2022.

(30) Foreign Application Priority Data
Dec. 28, 2021 (JP) 2021-215345
Dec. 22, 2022 (JP) 2022-205582

(51) Int. Cl. A61L 2/20; A61L 2/26; C01B 13/11; H05H 1/24

(52) U.S. Cl. CPC A61L 2/202; A61L 2/26; C01B 13/11; H05H 1/2406; A61L 2202/11; A61L 2202/13

(57) ABSTRACT

This active oxygen supply device is provided with a plasma actuator and an ozone decomposition device both arranged in a housing having at least one opening, in which the plasma actuator includes a first electrode, a dielectric body and a second electrode which are stacked in this order, the first electrode is an exposed electrode and blows out an ozone-containing induction flow in a first direction, the ozone decomposition device decomposes the ozone contained in the induction flow to generate active oxygen, the induction flow is converted to an induction flow containing the active oxygen, the edge part of the first electrode has a notched part having a shape enabling the increase in the flow rate of the induction flow, and the ozone decomposition device is at least one device selected from the group consisting of an ultraviolet light source and a heating device.

### CROSS-REFERENCE TO RELATED APPLICATION

This is a continuation of International Application No. PCT/JP 2022/048035, filed on Dec. 26, 2022, and designated the U.S., and claims priority from Japanese Patent Application No. 2021-215345 filed on Dec. 28, 2021, and Japanese Patent Application No. 2022-205582 filed on Dec. 22, 2022, the entire contents of which are incorporated herein by reference.

### BACKGROUND OF THE INVENTION

### Field of the Invention

The present invention is directed to an active oxygen supply device, a device for performing treatment with active oxygen, and a method for performing treatment with active oxygen.

### Description of the Related Art

As a means of sterilizing goods or the like, UV light and ozone are known. Japanese Patent Application Publication No. H01-025865 discloses, as a solution to a problem that sterilization using UV light is limited to a portion of an object to be sterilized that is irradiated with the UV light, a method that uses a sterilizing device having an ozone supply device, a UV generating lamp, and a stirring device to stir active oxygen generated by irradiating ozone with UV light generated from the UV generating lamp and sterilize even a shadow portion of a specimen.

### SUMMARY OF THE INVENTION

The present inventors examined sterilization performance provided by the sterilization method according to Japanese Patent Application Publication No. H01-025865, and found a case where the provided sterilization performance was approximately equal to sterilization performance provided by a conventional sterilization method using only ozone. While it was said that a sterilization capability of active oxygen intrinsically far exceeded a sterilization capability of ozone, such an examination result was unexpected.

At least one aspect of the present disclosure is directed to providing an active oxygen supply device which may more efficiently supply active oxygen to a surface of an object to be treated. Another aspect of the present disclosure is directed to providing a device for performing treatment with active oxygen which may more efficiently treat a surface of an object to be treated with the active oxygen.

Still another aspect of the present disclosure is directed to providing a method for performing treatment with active oxygen which may allow a surface of an object to be treated to be more efficiently treated with the active oxygen.

At least one aspect of the present disclosure provides an active oxygen supply device comprising, inside a housing having at least one opening portion:
a plasma actuator; and
an ozone decomposing device, wherein
the plasma actuator comprises a first electrode, a dielectric material, and a second electrode which are stacked in this order,
the first electrode is an exposed electrode provided on a first surface, which is one surface of the dielectric material,
when a voltage is applied between the first electrode and the second electrode, the plasma actuator generates a dielectric barrier discharge directed from the first electrode to the second electrode and cause an induced flow containing ozone to be blown out from the first electrode in a first direction, which is one direction along the surface of the dielectric material,
the ozone decomposing device decomposes the ozone contained in the induced flow to generate active oxygen in the induced flow, and the induced flow results in an induced flow containing the active oxygen,
the plasma actuator and the ozone decomposing device are arranged such that the induced flow containing the active oxygen flows out from the opening portion to the outside of the housing,
when the plasma actuator is seen through from the first electrode side, at least one portion of an edge portion of the first electrode leading in the first direction overlaps the second electrode,
when the plasma actuator is viewed from the first electrode side, the edge portion of the first electrode has a notched portion,
the notched portion has a shape which increases a flow rate of the induced flow compared to that in a case where the edge portion of the first electrode does not have the notched portion, and
the ozone decomposing device is at least one device selected from the group consisting of:
a UV light source that irradiates the induced flow containing the ozone with UV light to generate the active oxygen in the induced flow; and
a heating device that heats the induced flow containing the ozone to generate the active oxygen in the induced flow.

At least one aspect of the present disclosure provides a device for performing treatment with active oxygen, the device treating a surface of an object to be treated with the active oxygen and comprising, inside a housing having at least one opening portion:
a plasma actuator; and
an ozone decomposing device, wherein
the plasma actuator comprises a first electrode, a dielectric material, and a second electrode which are stacked in this order,
the first electrode is an exposed electrode provided on a first surface, which is one surface of the dielectric material,
when a voltage is applied between the first electrode and the second electrode, the plasma actuator generates a dielectric barrier discharge directed from the first electrode to the second electrode and cause an induced flow containing ozone to be blown out from the first electrode in a first direction, which is one direction along the surface of the dielectric material,
the ozone decomposing device decomposes the ozone contained in the induced flow to generate active oxygen in the induced flow, and the induced flow results in an induced flow containing the active oxygen,
the plasma actuator and the ozone decomposing device are arranged such that the induced flow containing the active oxygen flows out from the opening portion to the outside of the housing,
when the plasma actuator is seen through from the first electrode side, at least one portion of an edge portion of the first electrode leading in the first direction overlaps the second electrode,
when the plasma actuator is viewed from the first electrode side, the edge portion of the first electrode has a notched portion,
the notched portion has a shape which increases a flow rate of the induced flow compared to that in a case where the edge portion of the first electrode does not have the notched portion, and
the ozone decomposing device is at least one device selected from the group consisting of:
a UV light source that irradiates the induced flow containing the ozone with UV light to generate the active oxygen in the induced flow; and
a heating device that heats the induced flow containing the ozone to generate the active oxygen in the induced flow.

At least one aspect of the present disclosure provides a treatment method for treating a surface of an object to be treated with active oxygen, the treatment method comprising:
a step of preparing a device for performing the treatment with the active oxygen, wherein
the device for performing the treatment with the active oxygen comprises, inside a housing having at least one opening portion:
a plasma actuator; and
an ozone decomposing device,
the plasma actuator comprises a first electrode, a dielectric material, and a second electrode which are stacked in this order,
the first electrode is an exposed electrode provided on a first surface, which is one surface of the dielectric material,
when a voltage is applied between the first electrode and the second electrode, the plasma actuator generates a dielectric barrier discharge directed from the first electrode to the second electrode and cause an induced flow containing ozone to be blown out from the first electrode in a first direction, which is one direction along the surface of the dielectric material,
the ozone decomposing device decomposes the ozone contained in the induced flow to generate active oxygen in the induced flow, and the induced flow results in an induced flow containing the active oxygen,
the plasma actuator and the ozone decomposing device are arranged such that the induced flow containing the active oxygen flows out from the opening portion to the outside of the housing,
when the plasma actuator is seen through from the first electrode side, at least one portion of an edge portion of the first electrode leading in the first direction overlaps the second electrode,
when the plasma actuator is viewed from the first electrode side, the edge portion of the first electrode has a notched portion,
the notched portion has a shape which increases a flow rate of the induced flow compared to that in a case where the edge portion of the first electrode does not have the notched portion, and
the ozone decomposing device is at least one device selected from the group consisting of:
a UV light source that irradiates the induced flow containing the ozone with UV light to generate the active oxygen in the induced flow; and
a heating device that heats the induced flow containing the ozone to generate the active oxygen in the induced flow,
the treatment method further comprising:
a step of disposing the prepared device for performing the treatment with the active oxygen and the object to be treated at relative positions where a surface of the object to be treated is exposed to the induced flow when the induced flow is caused to flow out from the opening portion; and
a step of causing the induced flow to flow out from the opening portion to treat the surface of the object to be treated with the active oxygen.

According to at least one aspect of the present disclosure, it is possible to provide an active oxygen supply device which may more efficiently supply active oxygen to a surface of an object to be treated. In addition, according to the at least one aspect of the present disclosure, it is possible to provide a device for performing treatment with active oxygen which may more efficiently treat a surface of an object to be treated with the active oxygen. Furthermore, according to the at least one aspect of the present disclosure, it is possible to provide a method for performing treatment with active oxygen which may allow a surface of an object to be treated to be more efficiently treated with the active oxygen.

Further features of the present invention will become apparent from the following description of exemplary embodiments with reference to the attached drawings.

### BRIEF DESCRIPTION OF THE DRAWINGS

FIGS. 1A and 1B are a schematic cross-sectional view illustrating a configuration of an active oxygen supply device.

FIGS. 2A and 2B are a schematic diagram illustrating a configuration of a plasma actuator.

FIGS. 3A and 3B are an illustrative view of a shape of an edge portion of a first electrode and relative positions of the first electrode and a second electrode.

FIG. 4 is a schematic cross-sectional view illustrating a configuration of the active oxygen supply device.

FIG. 5 is a schematic diagram illustrating joining together of induced flows.

FIGS. 6A to 6I are an illustrative view of modifications of a shape of a notched portion of the edge portion of the first electrode.

FIGS. 7A to 7D are a schematic diagram illustrating overlap.

FIG. 8 is a diagram illustrating a method of measuring distances reached by the induced flows.

FIGS. 9A to 9F are a illustrative view of shapes of the electrodes used in an embodiment.

FIG. 10 is a diagram illustrating overlap between the electrodes.

FIGS. 11A and 11B are a schematic view of the active oxygen supply device.

FIG. 12 is a schematic view illustrating an embodiment of an active oxygen supply device including, as an ozone decomposing device, a UV light source and a humidifying device or the active oxygen supply device.

### DESCRIPTION OF THE EMBODIMENTS

In the present disclosure, statements of “from XX to YY” and “XX to YY” each representing a numerical value range mean numerical value ranges including lower limits and upper limits, which are endpoints, unless otherwise particularly specified. When numerical value ranges are stepwise stated, the upper and lower limits of the individual numerical value ranges can optionally be combined. In addition, in the present disclosure, such a statement as, e.g., “at least one selected from the group consisting of XX, YY, and ZZ” means any of XX, YY, ZZ, a combination of XX and YY, a combination of XX and ZZ, a combination of YY and ZZ, and a combination of XX, YY, and ZZ.

In the present disclosure, “treatment” of an object to be treated with active oxygen is assumed to include all types of treatment that can be carried out with the active oxygen, such as surface modification (hydrophilization treatment), sterilization, deodorization, and bleaching of a surface to be treated of an object to be treated with the active oxygen.

Further, “bacteria” as an object to be subjected to the “sterilization” according to the present disclosure refers to microorganisms, and the microorganisms include fungi, bacteria, unicellular algae, viruses, protozoa, and the like, as well as animal or plant cells (including stem cells, dedifferentiated cells, and differentiated cells), tissue cultures, fused cells obtained by genetic engineering (including hybridomas), dedifferentiated cells, and transformants (microorganisms). Examples of the viruses include norovirus, rotavirus, influenza virus, adenovirus, coronavirus, measles virus, rubella virus, hepatitis virus, herpes virus, HIV virus, and the like. Examples of the bacteria include Staphylococcus, Escherichia coli, Salmonella, Pseudomonas aeruginosa, Vibrio cholerae, Shigella, Anthrax, Mycobacterium tuberculosis, Clostridium botulinum, Tetanus, Streptococcus, and the like. Furthermore, examples of the fungi include Trichophyton, Aspergillus, Candida, and the like. Accordingly, the “sterilization” according to the present disclosure includes even virus inactivation.

In addition, the active oxygen in the present disclosure includes, for example, a free radical caused by decomposition of ozone (O₃), such as superoxide (·O₂⁻) or hydroxy radical (·OH).

Referring to the drawings, a mode for carrying out this disclosure will specifically be described below by way of example. Note that dimensions, materials, shapes, relative positioning, and the like of components described in the mode are to be appropriately changed depending on configurations of members to which the disclosure is applied and various conditions. In other words, it is not intended to limit the scope of this disclosure to the following modes. Furthermore, in the following description, components having the same functions are denoted by the same numerals in the drawings, and a description thereof may be omitted.

According to the study conducted by the present inventors, a reason for the limited sterilization capability of the sterilizing device according to Japanese Patent Application Publication No. H01-025865 is presumed as follows.

In Japanese Patent Application Publication No. H01-025865, the UV light is applied to the ozone to excite the ozone and generate the active oxygen having an extremely high sterilization power. The active oxygen mentioned herein is a general term for highly reactive active oxygen species such as superoxide anion radical ·O₂⁻ and hydroxy radical ·OH, which can immediately oxidatively decompose bacterium or viruses due to high reactivities thereof.

However, since ozone is extremely highly absorptive of UV light, it can be considered that, in the sterilizing device according to Japanese Patent Application Publication No. H01-025865, generation of active oxygen is limited to the vicinity of the UV generating lamp. In other words, it can be considered that the UV light does not satisfactorily reach the ozone present at a position away from the UV generating lamp and, at a place away from the UV generating lamp, the active oxygen is less likely to be generated.

In addition, the active oxygen is extremely unstable and, e.g., ·O₂⁻ having an extremely short half-life of 10⁻⁶ seconds and ·OH having an extremely short half-life of 10−9 seconds are considered to be rapidly converted to stable oxygen and water. Accordingly, it is conceivably difficult to passively fill the inside of a main body of the sterilizing device with the active oxygen generated in the vicinity of the UV generating lamp. In other words, it can be considered that the sterilization using the sterilization method according to Japanese Patent Application Publication No. H01-025865 is substantially performed with the ozone. Therefore, it can be considered that sterilization performance provided by the sterilization method according to Japanese Patent Application Publication No. H01-025865 is approximately equal to sterilization performance provided by the conventional sterilization method using only ozone.

Through such a consideration, the present inventors have recognized that, in treating the object to be treated by using the active oxygen, it is necessary to more positively place the object to be treated and a surface to be treated in an active oxygen atmosphere. Then, as a result of conducting study on the basis of such recognition, the present inventors have found that, with the active oxygen supply device and the device for performing treatment with active oxygen in the present disclosure described below, it is possible to allow the active oxygen to reliably reach the object to be treated in a state where a treatment capability thereof is maintained. As a result, the present inventors have found that it is possible to more positively place the object to be treated in the active oxygen atmosphere and significantly improve the efficiency of treating the object to be treated.

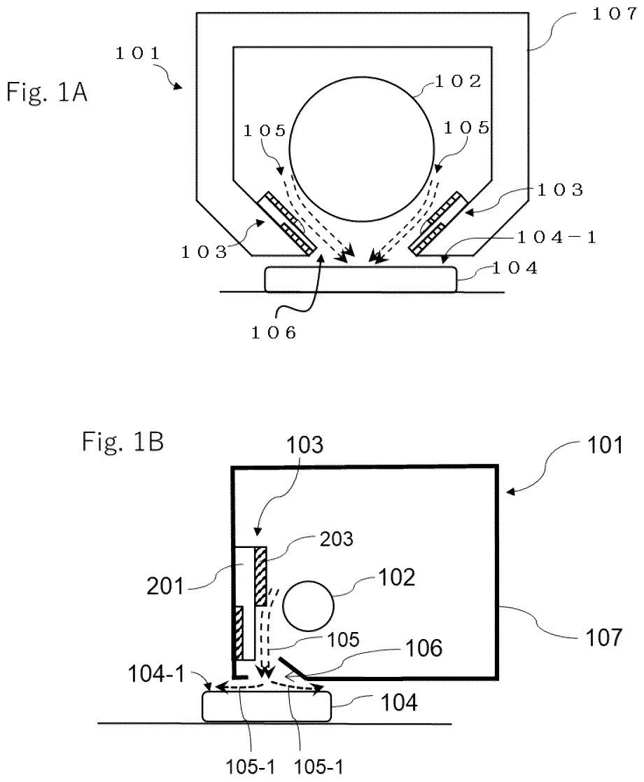

Using FIG. 1A, a description will be given below of an active oxygen supply device (device for performing treatment with active oxygen) 101 according to an embodiment of the present disclosure. The active oxygen supply device 101 according to the embodiment of the present disclosure includes, inside a housing 107 having at least one opening portion 106, a UV light source 102 serving as the ozone decomposing device 102 and a plasma actuator 103.

The UV light source serving 102 as the ozone decomposing device irradiates induced flows 105 with UV light to generate active oxygen in the induced flows 105. In FIG. 1A, a reference sign 104 denotes a to-be-treated object.

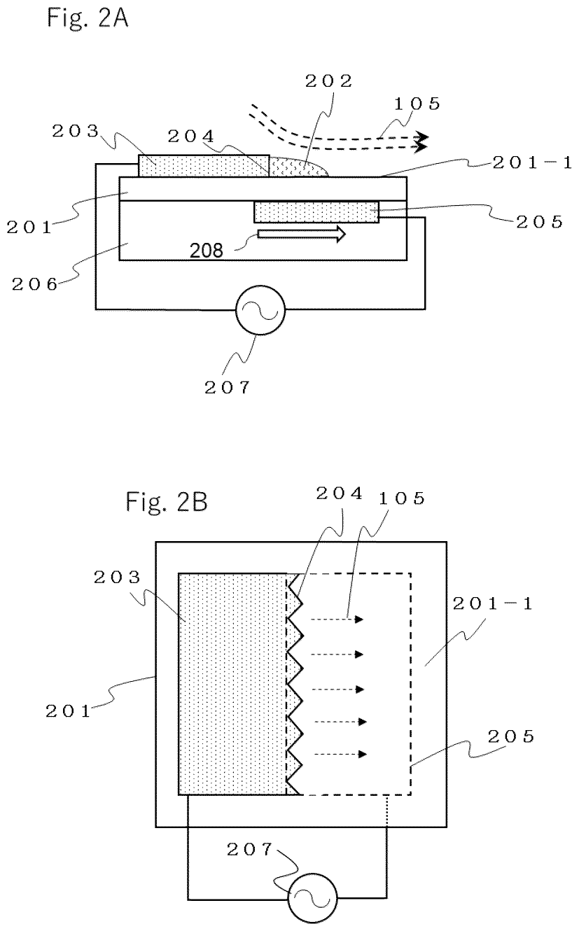

FIG. 2A illustrates a cross-sectional structure of an embodiment of the plasma actuator 103. The plasma actuator is a so-called DBD (Dielectric Barrier Discharge) plasma actuator (which may be hereinafter referred to simply as the “DBD-PA”) in which, on one surface (hereinafter referred to also as the “first surface”) of a dielectric material 201, a first electrode 203, which is an exposed electrode having an exposed end surface, is provided while, on a surface (hereinafter referred to also as the “second surface”) opposite to the first surface, a second electrode 205 is provided. In FIG. 2A, a reference sign 206 denotes a dielectric substrate in which the second electrode 205 is to be embedded in a direction of a thickness of the plasma actuator so as to prevent induced flows from an end surface of the second electrode from being formed. Between the first electrode and the second electrode, a voltage can be applied by a power source 207.

In the plasma actuator 103, the first electrode 203 and the second electrode 205, which are arranged with the dielectric material 201 being interposed therebetween, are positioned to be displaced from each other, while diagonally facing each other. By applying the voltage from the power source 207 between these electrodes (between the two electrodes), a dielectric barrier discharge directed from the first electrode 203 to the second electrode 205 is generated. Then, in a direction (an arrow 208 in FIG. 2A) in which the second electrode extends, a jet-stream flow resulting from a plasma 202 is induced from an edge portion 204 of the first electrode 203 along an exposed portion (portion uncovered with the first electrode) 201-1 of the first surface of the dielectric material 201.

At the same time, an air intake flow directed from a space inside a container to the electrodes is also generated. Electrons in the surface plasma 202 collide with oxygen molecules in air to dissociate the oxygen molecules and cause oxygen atoms. The resulting oxygen atoms collide with undissociated oxygen molecules to generate ozone. Consequently, by the action of the jet-stream flow resulting from the surface plasma 202 and the air intake flow, the induced flows 105 containing high-concentration ozone are generated from the edge portion 204 of the first electrode 203 along the surface of the dielectric material 201.

The plasma actuator 103 and the ozone decomposing device 102 are arranged such that the induced flows 105 containing active oxygen flow out from the opening portion 106 to the outside of the housing 107 to be supplied to a to-be-treated surface 104-1 of the to-be-treated object 104.

In other words, in the plasma actuator, the first electrode 203, the dielectric material 201, and the second electrode 205 are stacked in this order, and the first electrode 203 is the exposed electrode provided on the first surface of the dielectric material 201. When a voltage is applied between the first electrode 203 and the second electrode 205, the plasma actuator generates the dielectric barrier discharge directed from the first electrode 203 to the second electrode 205 and cause the induced flows to be blown out from the first electrode 203 in the first direction (direction of the arrow 208 in FIG. 2A), which is the one direction along the first surface of the dielectric material 201.

More specifically, the dielectric barrier discharge directed from the one edge portion 204 of the first electrode 203 to the second electrode 205 is caused, and the induced flows, which are unidirectional jet streams, are blown out from the one edge portion 204 of the first electrode 203 in the first direction (direction of the arrow 208 in FIG. 2A) along the first surface of the dielectric material 201.

Meanwhile, in a cross section along a thickness direction of the plasma actuator, the second electrode 205 is present to extend in the direction (first direction) in which the induced flows are blown out.

More specifically, for example, the plasma actuator has the dielectric material 201 and, when the cross section along the thickness direction of the plasma actuator is viewed, the first electrode 203 and the second electrode 205 are arranged to obliquely face each other via the dielectric material 201 in the thickness direction of the plasma actuator. The first electrode 203 is provided so as to cover a portion of the first surface of the dielectric material 201, and the first surface of the dielectric material has the exposed portion 201-1 uncovered with the first electrode 203.

FIG. 2B is a diagram when the plasma actuator is seen through from the first surface side of the dielectric material. At least one portion of the exposed portion 201-1 and the second electrode 205 indicated by a broken line have overlap therebetween. Consequently, the overlap between the at least one portion of the exposed portion and the second electrode corresponds to a region formed by an upper side, a lower side, and a right side of the broken line indicating the electrode 205 in FIG. 2B and by the edge portion 204.

By applying the voltage between the first electrode and the second electrode, from the edge portion 204 of the first electrode 203 leading in the first direction in the cross section (FIG. 2A) along the thickness direction, the induced flows containing the ozone are generated along the exposed portion of the dielectric material overlapping the second electrode 205.

The induced flows result in, e.g., wall surface jet streams along the exposed portion 201-1 to easily supply the high-concentration ozone to specified positions. A length (i.e., a length from the edge portion 204 of the first electrode leading in the first direction to an end portion of the first surface of the dielectric material) of the exposed portion 201-1 in an induced flow direction is not particularly limited, but is preferably 0.1 to 50 mm, more preferably 0.5 to 20 mm, or still more preferably 1.0 to 10 mm. As the length is larger, the plasma 202 extends longer, and the induced flows reach longer distances. Meanwhile, when the length is extremely large, a distance to the opening portion 106 increases. Therefore, the above ranges are preferred.

The UV light source 102 serving as the ozone decomposing device 102 irradiates the induced flows 105 with the UV light to decompose the ozone in the induced flows 105 and cause active oxygen in the induced flows. As illustrated in FIG. 1A, the plasma actuator 103 and the UV light source 102 are arranged such that the induced flows 105 containing the active oxygen flow out from the opening portion 106 to the outside of the housing 107 to be supplied to the to-be-treated surface 104-1 of the to-be-treated object 104.

Note that, in FIG. 1A, the UV light from the UV light source 102 also irradiates the surface of the to-be-treated object 104. In this case, even when the ozone in the induced flows 105 is not entirely decomposed into the active oxygen in the active oxygen supply device, the ozone that has reached the surface of the to-be-treated object 104 is decomposed in situ by the UV light to result in the active oxygen, and accordingly an improved treatment efficiency can be expected.

However, in the active oxygen supply device according to the present disclosure, the irradiation of the to-be-treated object with the UV light from the UV light source is not an essential configuration. For example, as illustrated in FIG. 1B, the plasma actuator having such a configuration that the UV light source 102 cannot directly be visually recognized from the opening portion 106 is also within the scope of the present disclosure. In the plasma actuator according to FIG. 1B, as a result of the decomposition of the ozone by the UV light from the UV light source 102, an induced flow 105-1 containing the active oxygen flows out from the opening portion 106 to be supplied to the to-be-treated surface 104-1 of the to-be-treated object 104.

In other words, in the active oxygen supply device according to the embodiment of the present disclosure, the induced flows 105 containing the ozone from the plasma actuator (plasma generating device) 103 flow out from the opening portion 106 to the outside of the housing 107 to be supplied to the to-be-treated surface 104-1 of the to-be-treated object 104, and the ozone decomposing device 102 decomposes the ozone (e.g., the UV light source 102 irradiates the induced flows 105 with the UV light) to generate the active oxygen in the induced flows 105 and thereby allow the active oxygen to be positively supplied to a region in the vicinity of the to-be-treated surface 104-1, specifically a space region (hereinafter referred to also as the “surface region”) at a height up to, e.g., about 1 mm from the to-be-treated surface.

Accordingly, the active oxygen can be supplied to the surface of the to-be-treated object before the generated active oxygen is converted to oxygen and water. Consequently, the to-be-treated surface 104-1 of the to-be-treated object 104 is reliably treated with the active oxygen.

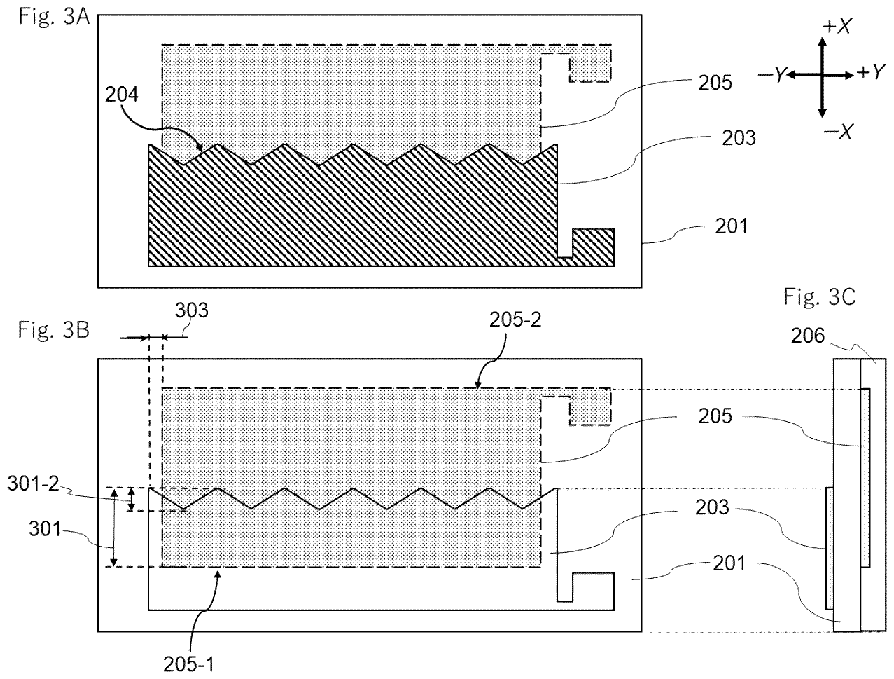

FIG. 3A illustrates a plan view resulting from observation from the first electrode 203 side when it is assumed that the dielectric material 201 of the plasma actuator 103 is transparent. In FIG. 3A, an X-axis is an axis parallel to the direction (first direction) in which the induced flows 105 from the plasma actuator 103 are blown out, and the first direction is a +X-direction. Meanwhile, a Y-axis is an axis perpendicular to the X-axis and, in FIG. 3A, a direction extending to a right side is a +Y-direction, while a direction extending to a left side is a-Y-direction.

The first electrode 203 is provided on the first surface of the dielectric material 201 to cover a portion of the surface of the dielectric material 201. In addition, as illustrated in FIG. 3A, the edge portion 204 of the first electrode leading in the first direction has a notched portion having a shape in which a width increases with approach to a +X-side in the first direction. Specifically, the edge portion 204 is notched into isosceles triangular shapes in which the +X-side in the first direction corresponds to bottom sides.

Alternatively, the plasma actuator may also be a so-called three-electrode plasma actuator in which a third electrode is further provided downstream in the direction in which the induced flows are blown out from the first electrode and on the first surface of the dielectric material 201. In this case, for example, it is possible to apply an ac voltage by using the first electrode as an AC electrode and apply a dc voltage by using the third electrode as a DC electrode. By applying a negative DC voltage to the DC electrode, it is also possible to generate a sliding discharge.

FIG. 3B is a perspective view obtained by viewing the plasma actuator 103 from the first electrode 203 side thereof. As illustrated in FIG. 3B and FIG. 3C, an edge portion 205-1 of the second electrode 205 leading in the −X-direction, which is embedded in the thickness direction of the plasma actuator 103, is located in the −X-direction to be ahead of a most leading position in the +X-direction of the edge portion 204 of the first electrode 203 leading in the +X-direction. In other words, the first electrode 203 and the second electrode 205 overlap each other by a length 301 in an X-axis direction. It can also be said that, in other words, as illustrated in FIG. 2A which is a cross-sectional view of the plasma actuator 103 along the thickness direction thereof, the first electrode 203 and the second electrode 205 are arranged to obliquely face each other with the dielectric material being interposed therebetween in the thickness direction of the plasma actuator 103.

Note that the edge portion 205-1 of the second electrode 205 leading in the −X-direction preferably has no notched portion. For example, the edge portion 205-1 has a linear shape. The shape of the second electrode is not particularly limited, and preferably has a quadrilateral shape such as, e.g., a rectangular shape or a square shape. By having a rectangular shape, it is possible to allow uniform induced flows to be generated.

It is assumed that the edge portion 204 of the first electrode leading in the first direction is an edge portion A and the edge portion 205-1 of the second electrode leading in the second direction (−X-direction) reverse to the first direction is an edge portion B. The length 301 of overlap between a most leading side of the notched portion of the edge portion A in the first direction (+X-direction) and the edge portion B may be hereinafter referred to as the “overlap amount”. Additionally, a length 301-2 between the most leading side of the notched portion of the edge portion A in the first direction (+X-direction) and a most leading side thereof in the second direction (−X-direction) may be hereinafter referred to as the “depth of the notched portion”. The depth of the notched portion is not particularly limited, but is preferably 0.1 mm to 10 mm, more preferably 0.2 mm to 5 mm, or still more preferably 0.5 mm to 3 mm.

On the assumption that the first electrode and the second electrode are electrically insulated from each other, as the shortest distance is smaller, the dielectric barrier discharge is more likely to occur. Accordingly, a thickness of a dielectric material portion of the dielectric material 201 which is present between the first electrode 203 and the second electrode 205 is preferably smaller within a range which does not cause dielectric breakdown when a voltage applied to the two electrodes. Specifically, when the applied voltage has an ac maximum-minimum voltage difference of 0.1 kVpp to 100 kVpp, the thickness of the dielectric material portion can be set to 10 μm to 1000 μm, or preferably to 10 μm to 200 μm. Meanwhile, the shortest distance between the first electrode and the second electrode is preferably 200 μm or less, or more preferably 100 μm to 200 μm.

The overlap amount between the first electrode and the second electrode is preferably set to, e.g., a value in excess of 0 μm and not more than 10000 μm, or particularly preferably to a value from 100 μm to 2000 μm.

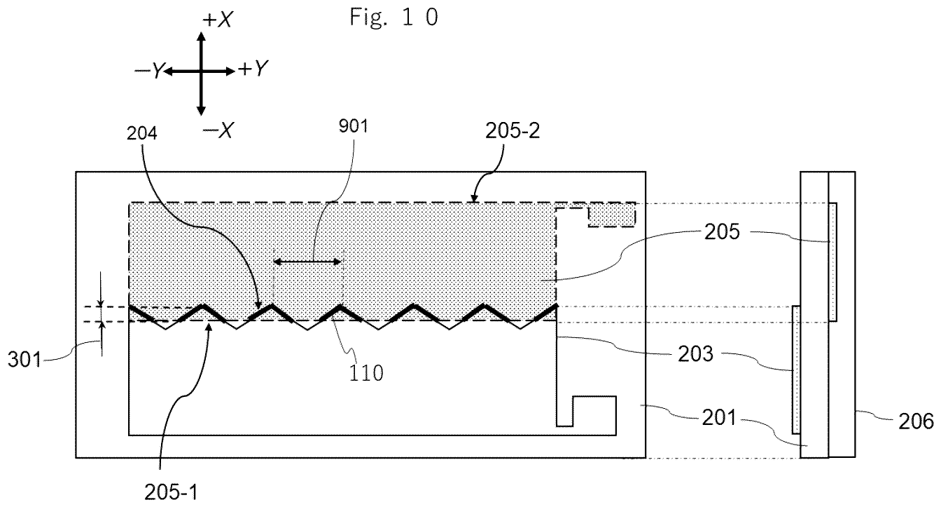

In the plasma actuator, when the notched portion of the first electrode does not entirely overlap the second electrode, the edge portion 204 of the first electrode having the notched portion, which is leading in the first direction, has portions (thick-line portions 110 in FIG. 10) overlapping the second electrode, and a total sum of lengths of the overlapping portions is L¹. L¹ is a length in the direction (in a plane including the X-direction and the Y-direction) along the surface of the dielectric material, which may be equivalent to a discharge length when a voltage is applied to the plasma actuator. Meanwhile, when it is assumed that the edge portion 204 of the first electrode leading in the first direction has no notched portion (i.e., the electrode is present even on the most leading side of the notched portion in the +X-direction, and the edge portion 204 has a linear shape), a length of a portion of the edge portion 204 which overlaps the second electrode in the direction (Y-axis direction) along the surface of the dielectric material is L².

At this time, L¹/L² is preferably 1.0 to 5.0, more preferably 1.2 to 4.0, still more preferably 1.5 to 3.0, and further more preferably 2.0 to 2.5. By being in the foregoing range, the L¹/L² can further increase speeds of the induced flows. The L¹/L² can be controlled by using the shape of the notched portion or a degree to which the notched portion is caused to overlap the second electrode.

Preferably, an edge portion 205-2 of the second electrode 205 which is leading in the +X-direction is present in the +X-direction to be ahead of the edge portion 204 of the first electrode 203 which is leading in the +X-direction. The second electrode present to extend in the +X-direction to be ahead of the edge portion 204 of the first electrode 203 can further increase directivities of the induced flows 105 in the +X-direction.

Then, as illustrated in FIG. 3A and FIG. 3B, the edge portion 204 of the first electrode 203 has the notch having isosceles triangular shapes having bottom sides leading in the +X-direction. Meanwhile, the second electrode 205 is provided to overlap the first electrode with the dielectric material being interposed therebetween, while extending in the +X-direction. By applying the voltage between these first electrode 203 and second electrode 205, it is possible to allow the intenser induced flows 105 to be generated from the edge portion 204 of the first electrode of the plasma actuator 103 in the +X-direction.

The following is a reason why the induced flows are intensified by providing the edge portion 204 with such a notch as to increase in width in the +X-direction as described above, which is assumed by the present inventors.

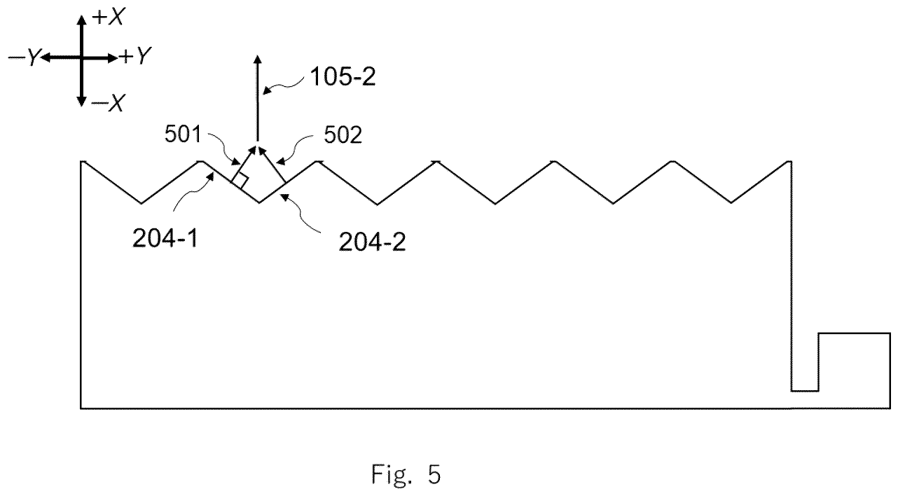

By applying the voltage between the first electrode and the second electrode, as illustrated in FIG. 5, an induced flow is generated from a side 204-1 forming the notch portion of the edge portion 204 having the isosceles triangular shapes in a direction of an arrow 501 perpendicular to the side. Meanwhile, from a side 204-2, an induced flow is generated in a direction of an arrow 502 perpendicular to the side. Then, the induced flow blown out in the direction of the arrow 501 and the induced flow blown out in the direction of the arrow 502 join together to generate an intense induced flow 105-2 containing ozone and blown out in the +X-direction.

Then, by irradiating such an induced flow with the UV light from the UV light source, the ozone in the induced flow is decomposed into active oxygen, and the active oxygen is contained in the induced flow. According to the study conducted by the present inventors, it can be considered that the active oxygen contained in such an induced flow can maintain an active state thereof over a period longer than a generally termed lifetime of active oxygen (half-life of ·O₂⁻: 10⁻⁶ seconds, half-life of ·OH: 10⁻⁹ seconds). A conceivable reason for this may be that the active oxygen in the induced flow is protected in the regulated induced flow, collision with another active species or molecules in atmospheric air is suppressed, and deactivation resulting from a reaction is unlikely to occur.

As a result, the active oxygen supply device according to the present disclosure can more reliably supply a larger amount of the active oxygen to the object to be treated and further improve the efficiency of the treatment of the object to be treated.

Materials for forming the first electrode and the second electrode are not particularly limited as long as the materials have excellent conductivities. For example, a metal such as copper, aluminum, stainless steel, gold, silver, or platinum, a plated or vapor-deposited metal, a conductive carbon material such as carbon black, graphite, or carbon nanotube, a composite material obtained by mixing the conductive carbon material with a resin or the like can be used. The material forming the first electrode and the material forming the second electrode may be the same or different.

Among these, the material forming the first electrode is preferably aluminum, stainless steel, or silver from viewpoints of avoiding corrosion of the electrodes and uniformizing discharge. For the same reason, the material forming the second electrode is also preferably aluminum, stainless steel, or silver.

As shapes of the first electrode and the second electrode, a flat plate shape, a wire shape, a needle shape, and the like can be used without particular limitation. The shape of the first electrode is preferably a flat plate shape. Meanwhile, the shape of the second electrode is preferably a flat plate shape. When at least one of the first electrode and the second electrode has a flat plate shape, an aspect ratio (Length of Long Side/Length of Short Side) of the flat plate is preferably 2 or more.

The dielectric material is not particularly limited as long as the dielectric material is a material having a high electrically insulating property. For example, resins such as polyimide, polyester, a fluorine resin, a silicone resin, an acrylic resin, and a phenol resin, glass, ceramics, composite materials obtained by mixing these with resins or the like, and the like can be used. Among these, a ceramic, glass, or a silicone resin is preferably used from viewpoints of a strength and an insulating property. In particular, a silicone resin, which is flexible, can increase a degree of freedom of the plasma actuator shape.

A thickness of the electrode is not particularly limited whether the electrode is the first electrode or the second electrode, but can be set to 10 μm to 1000 μm. When the thickness is 10 μm or more, a resistance decreases, and the plasma is likely to be generated. When the thickness is 1000 μm or less, electric field concentration is likely to occur, and consequently the plasma is likely to be generated.

A length (electrode width) of the electrode along the first direction (X-axis direction) is not particularly limited whether the electrode is the first electrode or the second electrode, but can be set to 1000 μm or more.

When the edge portion of the second electrode is exposed, it may be possible that the plasma is generated also from the edge portion of the second electrode, and induced flows directed opposite to the induced flows 105 derived from the first electrode are formed. In the active oxygen supply device according to the present embodiment, it is preferable to set an ozone concentration in an inner space of the active oxygen supply device other than the surface region of the object to be treated as low as possible. In addition, it is preferable not to cause flowing motion of a gas that may disturb the induced flows 105 in a container. Accordingly, it is preferable not to cause induced flows derived from the second electrode. Therefore, the second electrode 205 is preferably covered with a dielectric material such as the dielectric substrate 206 or embedded in the dielectric material 201 as illustrated in FIG. 2A and FIG. 3C for prevention of generation of the plasma from the edge portion of the second electrode or the like.

The second electrode needs only to be embedded to such a degree as to be able to prevent the plasma from being generated from the edge portion of the second electrode, and it may also be possible that, e.g., a portion of the surface of the second electrode is exposed, and the exposed surface of the second electrode and the dielectric substrate 206 or the dielectric material 201 form the same plane. Preferably, the edge portion of the second electrode is covered with the dielectric substrate 206 or the dielectric material 201. Accordingly, for example, the plasma actuator is preferably a SDBD (single dielectric barrier discharge) plasma actuator.

The induced flows 105 containing the high-concentration ozone flow in a direction of the jet-stream flow resulting from the surface plasma extending from the edge portion 204 of the first electrode 203 along the exposed portion 201-1 of the first surface of the dielectric material 201, i.e., a direction extending from the edge portion 204 of the first electrode 203 along the exposed portion 201-1 of the first surface of the dielectric material. The induced flows are flows of a gas containing the high-concentration ozone and having a speed of about several meters per second to several tens of meters per second.

The voltage to be applied between the first electrode 203 and the second electrode 205 of the plasma actuator is not particularly limited as long as the voltage is in an embodiment which allows the plasma actuator to generate a plasma. The voltage may be either a dc voltage or an ac voltage, but is preferably the ac voltage. Using a pulse voltage as the voltage is also a preferred embodiment.

Furthermore, an amplitude and a frequency of the voltage can be set appropriately to adjust flow rates of the induced flows and ozone concentrations in the induced flows. In this case, a selection may be made appropriately from a viewpoint of generating, in each of the induced flows, an ozone concentration required to generate an effective active oxygen concentration or effective active oxygen amount according to an object of the treatment, supplying the generated active oxygen to the surface region of the object to be treated in a state where the effective active oxygen concentration or effective active oxygen amount according to the object of the treatment are maintained, or the like.

For example, the amplitude of the voltage can be set to 1 kV to 100 kV. In addition, the frequency of the voltage can be set preferably to 1 kHz or more, or more preferably to 10 kHz to 100 kHz.

When the ac voltage is used as the voltage, a waveform of the ac voltage is not particularly limited, and a sine wave, a rectangular wave, a triangular wave, or the like can be used but, from a viewpoint of a rising speed of the voltage, the rectangular wave is preferred.

A duty ratio of the voltage can also be selected appropriately, but the rising speed of the voltage is preferably high. Preferably, the voltage is applied such that the voltage rises from a bottom of the amplitude of a wavelength to reach a top thereof at 10,000,000 V/second or more.

Note that a value (voltage/film thickness) obtained by dividing the amplitude of the voltage to be applied between the first electrode 203 and the second electrode 205 by a film thickness of the dielectric material 201 is preferably set to 10 kV/mm or more.

### <Ozone Decomposing Device>

The active oxygen supply device or an active oxygen treatment device includes the ozone decomposing device 102. The ozone decomposing device decomposes the ozone contained in the induced flows to generate active oxygen in the induced flows. As the ozone decomposing device, an ozone decomposing device that can act on the ozone contained in the induced flows and decompose the ozone can be used. As the ozone decomposing device, an ozone decomposing device that can decompose the ozone without disturbing the induced flows is preferred.

The ozone decomposing device is at least one device selected from the group consisting of a UV light source that irradiates the induced flows containing the ozone with UV light to generate active oxygen in the induced flows and a heating device that heats the induced flows containing the ozone to generate active oxygen in the induced flows. The ozone decomposing device may also be a combination thereof. For example, the UV light source that irradiates the induced flows with the UV light and the heating device that heats the induced flows may also be used in combination. As the ozone decomposing device, at least the UV light source is used more preferably. Alternatively, the ozone decomposing device may also include both of at least one selected from the above group consisting of the UV light source and the heating device and a humidifying device that humidifies the induced flows containing the ozone to generate active oxygen in the induced flows.

A description will be given below of each of the devices.

### <UV Light Source and UV Light>

The UV light source is not particularly limited as long as the UV light source can emit UV light that can excite the ozone and generate active oxygen. The UV light source is also not particularly limited as long as the UV light source has a wavelength and an illuminance of the UV light which are required to excite the ozone and obtain the effective active oxygen concentration or effective active oxygen amount according to the object of the treatment.

For example, a light absorption spectrum of the ozone has a peak value of 260 nm, and accordingly a peak wavelength of the UV light is preferably 220 nm to 310 nm, more preferably 253 nm to 285 nm, or still more preferably 253 nm to 266 nm.

As a specific UV light source, a low-pressure mercury lamp in which mercury is encapsulated together with an inert gas, such as argon or neon, in quartz glass, a cold cathode tube UV lamp (UV-CCL), a UV LED, or the like can be used. Wavelengths of the low-pressure mercury lamp and the cold cathode tube UV lamp may appropriately be selected from among 254 nm and the like. Meanwhile, from a viewpoint of output performance, a wavelength of the UV LED may appropriately be selected from among 265 nm, 275 nm, 280 nm, and the like.

### <Heating Device>

The heating device 102 is not particularly limited as long as the heating device 102 can give thermal energy that can excite the ozone in the induced flows and generate active oxygen. Since thermal decomposition of the ozone begins at about 100° C., a device that can heat the induced flows to about 120° C. is preferred. Meanwhile, when 120° C. is exceeded, the object to be treated may incur thermal deterioration such as melting or decomposition, and accordingly a temperature is preferably 200° C. or less. The temperature is preferably 100 to 140° C., or more preferably 110 to 130° C.

The heating device is not particularly limited and, e.g., a ceramic heater, a cartridge heater, a sheathed heater, an electric heater, an oil heater, or the like can be used. In the case of a device including a metallic heat generator, the heat generator is preferably a material having an excellent oxidation resistance, such as a nichrome-based alloy or tungsten. Preferably, the cartridge heater is used.

### <Humidifying Device>

The humidifying device 102 is not particularly limited as long as the humidifying device 102 can generate active oxygen in the induced flows by humidifying the inside of the housing to cause the induced flows to contain water and decomposing the ozone in the induced flows with the water. The humidification mentioned herein is giving moisture to a target, and an embodiment of the moisture is not particularly limited and may also be at least one selected from the group consisting of a gas, a liquid, and a solid. As the water to be used when the moisture is given, known water may optionally be used, and a substance other than water may also be contained.

The humidifying device is not particularly limited and, for example, a vaporization-type humidifying device or a mist-type humidifying device can be used.

Preferably, the humidifying device has a directivity with respect to a direction in which the moisture is supplied (hereinafter also referred to simply as the directivity) so as not to increase a humidity in the vicinity of the plasma actuator. By having the directivity, the humidifying device can efficiently humidify the vicinities of the induced flows or the vicinity of the surface of the object to be treated without increasing the humidity in the vicinity of the plasma actuator.

To allow the humidifying device to have the directivity, a known method can be used appropriately. For example, a method in which, by providing a fan and thereby generating an air flow, the moisture is transferred in a direction of the air flow, a method in which an appropriate pressure is given to the moisture by using an air pump or the like to eject the moisture in an intended direction, or the like can be used. To prevent the induced flows from being disturbed, it is preferable to direct the humidifying device in the same direction (first direction) as the direction of each of the induced flows.

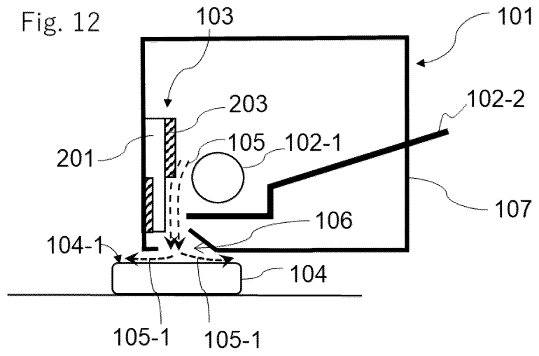

FIG. 12 illustrates an example of the active oxygen supply device or active oxygen treatment device according to an embodiment of the present disclosure which includes, as the ozone decomposing device 102, a UV light source 102-1 that irradiates the induced flows 105 including the ozone with UV light to generate active oxygen in the induced flows 105 and a humidifying device 102-2 that humidifies the induced flows 105 to generate active oxygen in the induced flows.

For example, the humidifying device 102-2 supplies moisture from the end portion thereof on the induced flow 105 side (left side in the drawing) in the drawing to the induced flows.

### <Arrangement of Plasma Actuator, Ozone Decomposing Device, and Object to be Treated>

In the active oxygen supply device 101, a position of the plasma actuator 103 that causes the induced flows containing the ozone is not particularly limited as long as the plasma actuator is positioned such that, due to the UV light emitted from the UV light source 102, which is the ozone decomposing device, the induced flows 105 flow out from the opening portion to the outside of the housing to be supplied to the surface of the object to be treated in a state where the effective active oxygen concentration or effective active oxygen amount according to the object of the treatment is maintained. The same applies also to a case where the ozone decomposing device is the heating device or the humidifying device.

For example, the plasma actuator and the ozone decomposing device may appropriately be arranged such that the induced flows 105 containing the generated active oxygen are supplied at shortest distances to the surface of the object to be treated.

Alternatively, for example, the plasma actuator may appropriately be positioned such that the to-be-treated surface 104-1 of the to-be-treated object is included in an extension line of a direction extending from the edge portion 204 of the first electrode 203 of the plasma actuator leading in the first direction along the first surface (the exposed portion 201-1 thereof) of the dielectric material. For example, the extension line is preferably in contact with the to-be-treated surface 104-1.

In addition, an extension line of the direction (the same as the +X-direction) extending from the edge portion of the first electrode 203 of the plasma actuator leading in the first direction along the first surface of the dielectric material is preferably directed to face the opening portion. This allows the induced flows to easily flow out from the opening portion to the outside of the housing.

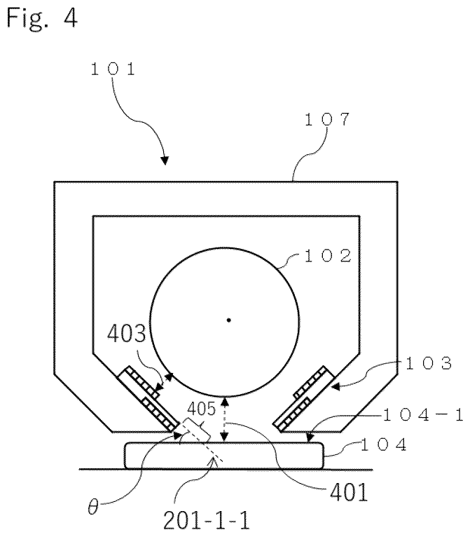

It is assumed that, when the opening portion of the active oxygen supply device is directed to face vertically downward, a narrow angle formed between an extension line 201-1-1 of a direction extending from the edge portion of the first electrode of the plasma actuator along the exposed portion 201-1 of the first surface of the dielectric material and a horizontal plane (plane perpendicular to a vertical direction) is θ (hereinafter referred to also as the plasma actuator incidence angle or PA incidence angle seen in FIG. 4). The narrow angle θ is not particularly limited as long as the narrow angle θ is an angle which allows the induced flows to be positively supplied to the surface region of the object to be treated in the state where effective active oxygen concentration or the effective active oxygen amount according to the object of the treatment is maintained or an angle which allows the treatment to be performed with the active oxygen, but is preferably θ° to 90°, or more preferably 30° to 70°.

By arranging the plasma actuator and the ozone decomposing device as described above, it is possible to locally supply the induced flows each containing the active oxygen and having a certain flow rate to a region of the object to be treated in the vicinity of the surface thereof or treat the region with the active oxygen. In addition, the induced flows flown out from the opening flow along the surface of the object to be treated, and a portion of the surface to be treated of the object to be treated which is other than a portion thereof facing the opening portion is also exposed to the induced flows containing the active oxygen. This allows a wider range of the to-be-treated surface 104-1 to be treated with the active oxygen.

In other words, the plasma actuator may appropriately be disposed such that the to-be-treated surface 104-1 of the to-be-treated object is included in an extension line of the first direction (direction in which the induced flows are blown out). It is assumed that, when the opening portion of the active oxygen supply device is directed to face vertically downward, a narrow angle formed between the first direction (direction in which the induced flows are blown out) and the horizontal plane (plane perpendicular to the vertical direction) is θ′. The angle θ′ is preferably θ° to 90°, or more preferably 30° to 70°.

The ozone decomposing device is otherwise not particularly limited as long as the ozone decomposing device is disposed so as to allow active oxygen to be generated in the induced flows and allow the surface of the object to be treated to be treated in a state where the effective active oxygen concentration or effective active oxygen amount according to the object of the treatment is maintained.

As described above, the induced flows containing the ozone are positively supplied to the region in the vicinity of the surface of the object to be treated. Additionally, when the ozone decomposing device is the UV light source, by irradiating the induced flows with the UV light, it is possible to generate active oxygen in the induced flows. Accordingly, through the irradiation of the induced flows with the UV light, the ozone is excited to allow the induced flows in a state where the active oxygen is generated to be positively supplied to the surface of the object to be treated and also allow the active oxygen concentration or active oxygen amount in the surface of the object to be treated to be significantly increased.

Relative positions of the ozone decomposing device and the plasma actuator are otherwise not particularly limited as long as the ozone decomposing device and the plasma actuator are arranged so as to allow active oxygen to be generated in the induced flows and allow the surface of the object to be treated to be treated in the state where the effective active oxygen concentration or effective active oxygen amount according to the object to the treatment is maintained.

Moreover, since a distance between the ozone decomposing device and the plasma actuator varies depending on the object of the treatment, the distance therebetween cannot generally be defined. For example, a distance between a surface of the dielectric material of the plasma actuator which faces the ozone decomposing device and the ozone decomposing device is set preferably to 10 mm or less, or more preferably to 4 mm or less. However, the plasma actuator need not be placed at a position within about 10 mm from the ozone decomposing device. As long as the active oxygen in the induced flows is allowed to have an effective concentration according to the object of the treatment in relation to an element that may decompose the ozone described later, such as the illuminance or wavelength of the UV light, the distance between the ozone decomposing device and the plasma actuator is not particularly limited.

Alternatively, providing at least one of the ozone decomposing device and the plasma actuator with a moving means and rendering at least one of the ozone decomposing device and the plasma actuator movable so as to cause a uniform degree of ozone decomposition is also a preferred embodiment.

Relative positions of the active oxygen supply device and the object to be treated are appropriate as long as at least one of the active oxygen supply device and the object to be treated is disposed such that active oxygen is generated in the induced flows, and the surface of the object to be treated is exposed to the induced flows in which the effective active oxygen concentration or effective active oxygen amount according to the object of the treatment is maintained.

When the ozone decomposing device is the UV light source, the UV light source may be either disposed at a position where the surface of the object to be treated can be irradiated with UV light or disposed at a position where the surface of the object to be treated cannot be irradiated with the UV light. Even when the surface of the object to be treated cannot be irradiated with the UV light from the UV light source, the treatment device using the active oxygen according to the present embodiment can treat the surface to be treated through the exposure of the surface to be treated to the active oxygen in the induced flows.

In the same manner as when the ozone decomposing device is the heating device also, the heating device may be either disposed at a position where the surface of the object to be treated can be heated or disposed at a position where the surface of the object to be treated cannot be heated.

In sterilization treatment using the UV light, what is sterilized is only the surface irradiated with the UV light. However, in the sterilization treatment using the active oxygen supply device according to the present disclosure, bacteria present at a position that can be reached by the active oxygen can be sterilized. Accordingly, for example, even bacteria present between fibers, which is hard to sterilize with UV light irradiation from the outside, may be sterilized.

Meanwhile, when the UV light source is disposed so as to allow the UV light therefrom to irradiate the surface of the object to be treated which is placed outside the housing via the opening portion, the undecomposed ozone present in the induced flows can be decomposed in situ on the surface to be treated to be able to generate active oxygen on the surface to be treated. As a result, it is possible to further increase a level of the treatment and the efficiency of the treatment.

In this case, the illuminance of the UV light at the surface of the object to be treated or the illuminance of the UV light in the opening portion is not particularly limited but, even at or in, e.g., the surface of the object to be treated or the opening portion also, it is preferable to set the illuminance of the UV light which can decompose the ozone contained in the induced flows, generate active oxygen in the induced flows, and provide the effective active oxygen concentration or effective active oxygen amount according to the object of the treatment. Specifically, for example, a specific example of the illuminance of the UV light at the surface of the object to be treated or the illuminance of the UV light in the opening portion is preferably 40 μW/cm² or more, more preferably 100 μW/cm² or more, still more preferably 400 μW/cm² or more, or particularly preferably 1000 μW/cm² or more. The upper limit of the illuminance is not particularly limited, but can be set to, e.g., 10000 μW/cm² or less.

Meanwhile, a distance between the ozone decomposing device and the surface of the object to be treated needs only to be adjusted depending on the object of the treatment and is not particularly limited, but is set preferably to, e.g., 10 mm or less, or more preferably to 4 mm or less in view of a lifetime of the active oxygen contained in the induced flows. However, the object to be treated need not be placed such that the surface to be treated of the object to be treated is present at a position within about 10 mm from the ozone decomposing device. As long as the active oxygen in the induced flows can be set to the effective concentration according to the object of the treatment in relation to the element that may decompose the ozone, such as the illuminance of the UV light, the distance between the ozone decomposing device and the object to be treated is not particularly limited.

An amount of ozone generated per unit time in the plasma actuator in a state where the ozone in the induced flows is kept from being decomposed by the ozone decomposing device is, e.g., preferably 15 μg/minute or more, or more preferably 30 μg/minute or more. An upper limit of the ozone generation amount is not particularly limited, but is, e.g., 1000 μg/minute or less. In other words, a preferred range is from 15 μg/minute to 1000 μg/minute.

Flow rates of the induced flows need only to be, e.g., speeds that allow the generated active oxygen to be positively supplied to the surface region of the object to be treated in the state where the effective active oxygen concentration or effective active oxygen amount according to the object of the treatment is maintained, which is, e.g., about 0.01 m/s to 100 m/s as described above.

As described above, the ozone concentration in the induced flows generated from the plasma actuator and the flow rates of the induced flows can be controlled by using the thicknesses or materials of the electrodes and the dielectric material, the type, amplitude, or frequency of the voltage to be applied, or the like.

### <Housing and Opening Portions>

The active oxygen supply device in the present disclosure includes the housing 107 having the at least one opening portion 106, and the ozone decomposing device 102 and the plasma actuator 103 each disposed in the housing.

The opening portion is not particularly limited as long as the opening portion is in an embodiment in which the induced flows 105 containing the active oxygen, which is caused by the plasma actuator 103 and the ozone decomposing device 102, flow to the outside of the housing 107. A size of the opening portion, a position of the opening portion, and relative positions of the opening portion and the object to be treated can appropriately be chosen such that, e.g., the generated active oxygen can positively be supplied to the surface region of the object to be treated in the state where the effective active oxygen concentration or effective active oxygen amount according to the object of the treatment is maintained.

In addition, a distance between the plasma actuator and the opening portion is such that, to allow the active oxygen in the induced flows to be more effectively used for the intended treatment, a distance between the plasma actuator and the object to be treated is preferably short. Accordingly, it is preferable to dispose the plasma actuator at a position closer to the opening portion. To protect the plasma actuator, it is also preferable to dispose the plasma actuator at a position set back from the opening portion. By way of example, it is preferable to dispose the plasma actuator on an inner wall of the housing such that an end portion of the plasma actuator which is closer to the opening portion is at 0.5 mm to 1.5 mm from an edge portion of the opening portion in the inner wall of the housing.

The active oxygen supply device in the present disclosure can be used not only for an application in which the object to be treated is sterilized, but also for all the applications that are implemented by supplying the active oxygen to the object to be treated. For example, the active oxygen supply device in the present disclosure can be used also for an application in which the object to be treated is deodorized, an application in which the object to be treated is bleached, hydrophilization surface treatment for the object to be treated, or the like.

Meanwhile, the device for performing treatment with active oxygen in the present disclosure can be used not only for treatment of sterilizing the object to be treated, but also for, e.g., treatment of deodorizing the object to be treated, treatment of bleaching the object to be treated, a surface treatment of hydrophilizing the object to be treated, or the like.

The present disclosure also provides a treatment method for treating a surface of an object to be treated with active oxygen, the treatment method comprising:
a step of preparing the device for performing treatment with active oxygen described above;
a step of disposing the prepared device for performing treatment with active oxygen and the object to be treated at relative positions where a surface of the object to be treated is exposed to an induced flow when the induced flow is caused to flow out from the opening portion; and
a step of causing the induced flow to flow out from the opening portion to treat the surface of the object to be treated with the active oxygen.

Note that, in the present disclosure, the “effective active oxygen concentration or effective active oxygen amount” refers to an active oxygen concentration or an active oxygen amount for attaining an object with respect to the object to be treated such as, e.g., sterilization, deodorization, bleaching, or hydrophilization, and can be adjusted appropriately according to the object by using the thicknesses and materials of the electrodes and the dielectric material which are included in the plasma actuator, the type, amplitude, and frequency of the voltage to be applied, a degree of ozone decomposition (the illuminance and irradiation time of the UV light, the heating temperature, the heating time, a humidification moisture amount, and a humidification time) by the ozone decomposing device, the PA incidence angle, or the like.

### Second Embodiment

In the active oxygen supply device according to the present disclosure, the notched portion of the edge portion 204 of the first electrode is not limited to the embodiment illustrated in FIG. 3A in which the shape of each of the isosceles triangular shapes having the bottom side on the +X side continues along the edge portion 204 in the −Y-direction to the +Y-direction (hereinafter referred to also as the “width direction”).

The notched portion needs only to have a shape that increases the flow rates of the induced flows compared to those in a case where the edge portion of the first electrode does not have the notched portion. For example, it is only necessary that the induced flows generated from the edge portion of the first electrode join together, and the notched portion is provided in a shape which allows the induced flows joined together to be easily blown out in the first direction (+X-direction). To provide the shape that increases the flow rates of the induced flows, a means of providing the notched portion such that vectors of the induced flows generated from the edge portion of the first electrode include components directed in the first direction or do not include components directed in the second direction or the like can be used.

A shape of the notched portion is preferably such that, when the plasma actuator is viewed from the first electrode side, a width of the notch increases in the first direction. This allows the induced flows joined together to be easily intensively blown out in the first direction.

The number of the notched portions is not particularly limited. For example, in the width direction, the notched portion may be provided at one position, may also be provided regularly, or may further be provided periodically. However, from a viewpoint of further intensifying the induced flows, the notched portions are preferably provided at a plurality of positions.

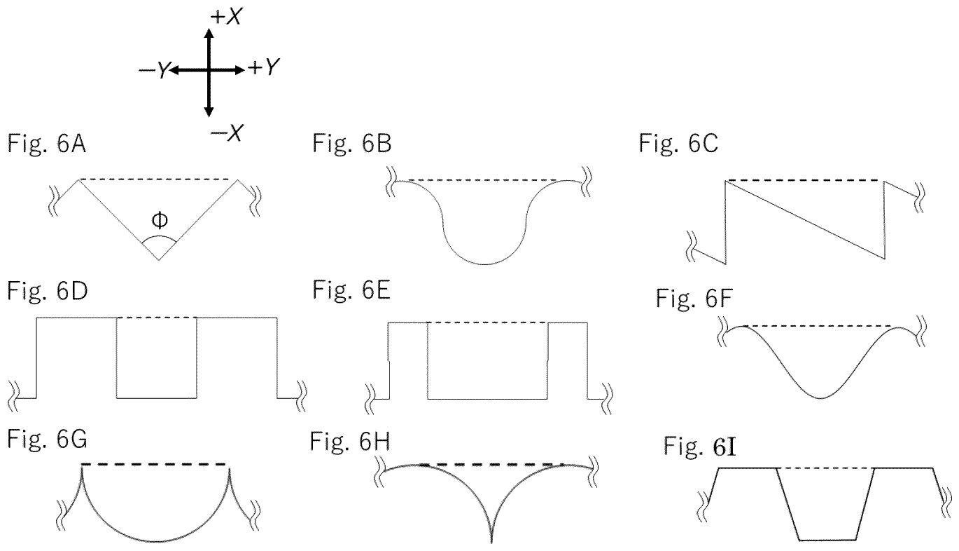

Furthermore, the shape of the notched portion is also not limited to the isosceles triangular shapes illustrated in FIG. 3A. In other words, the shape of the notched portion may be any shape as long as intensities of the induced flows in the +X-direction can be increased on the basis of a case where the edge portion 204 has no notched portion. FIG. 6A to FIG. 61 illustrate non-limitative modifications. The case where there is no notched portion means a case where the electrode is present even on the most leading side of the notched portion in the +X-direction, and the edge portion 204 is linear.

For example, the shape of the notched portion may also be a triangular shape as those in FIG. 3A and FIG. 6A, or a substantially triangular shape such that a corner of the triangular shape is rounded or a side thereof is partially rounded. When the notched portion has a triangular shape, from a viewpoint of generating intenser induced flows, an angle @ (inner angle formed on a side leading in the −X-direction by the edge portion 204) formed by the edge portion 204 in the notched portion illustrated in FIG. 6A is preferably 30° to 150°, more preferably 45° to 135°, or still more preferably 60° to 120°.

Alternatively, as illustrated in FIG. 6C, the shape of the notched portion may also be a sawtooth shape. As illustrated in FIG. 6B, the shape of the notched portion may also be an arc shape, an ellipsoidal arc shape, or a substantially arc shape in which the arc is partially deformed. The arc shape includes such shapes as those illustrated in FIG. 6G and FIG. 6H.

Still alternatively, as illustrated in FIG. 6D and FIG. 6E, the shape of the notched portion may also be a rectangular shape, or may also be a substantially rectangular shape in which a corner of a rectangle is rounded or a side thereof is partially rounded. When the notched portion has the rectangular shape, electric lines of force at apexes of right-angled portions of the ends of the notched portion leading in the +X-direction are in directions each at 135° with respect to both edge-portion line segments adjacent thereto, and the induced flows are blown in the directions of the electric lines of force, and accordingly it is possible to increase the intensities of the induced flows in the +X direction.

The shape of the notched portion may be a sine wave shape as that in FIG. 6F, or may also be a trapezoidal shape as that in FIG. 6I. Alternatively, the shape of the notched portion may also be a substantially trapezoidal shape in which a corner of a trapezoid is rounded or a side of the trapezoid is partially rounded.

As the shape of the notched portion, one of these shapes may be used alone or some of these shapes may be combined. In other words, the shape of the notched portion preferably has a triangular shape, a substantially triangular shape, a sawtooth shape, an arc shape, an ellipsoidal arc shape, a substantially arc shape, a sine wave shape, a trapezoidal shape, a substantially trapezoidal shape, a rectangular shape, or a substantially rectangular shape. The shape of the notched portion more preferably has the triangular shape, the arc shape, the sine wave shape, or the trapezoidal shape, or still more preferably has the triangular shape or the sine wave shape.

A shape of the edge portion has at least one notched portion. From the viewpoint of further intensifying the induced flows, a larger number of the notched portions are preferably present, and may be present intermittently, but are preferably present continuously. It is preferable that, of the edge portion of the first electrode, a portion that generates the induced flows does not have a side perpendicular to the first direction. This allows the entire edge portion to generate the intense induced flows.

The notched portion is preferably regularly present in the edge portion. In addition, the notched portion is preferably periodically present in the edge portion. More preferably, the notched portion has a regular and periodical waveform. As an example of the notched portion having the regular and periodical waveform, a waveform continuously having the shape described above is preferred. More preferably, the shape of the edge portion is the triangular wave shape or the sine wave shape.

As examples of one period of a waveform, FIG. 6A to FIG. 6I are shown by way of example. Twice an amplitude (depth of the notch in the X-direction) and a wavelength (length of the first electrode edge portion in the Y-direction) of this one-period waveform are not particularly limited. As twice the amplitude, 0.1 mm to 10 mm are preferable, 0.2 mm to 3 mm are more preferable, and 0.3 mm to 2 mm are further preferable. As the wavelength, 0.1 mm to 10 mm are preferable, 0.5 mm to 5 mm are more preferable, and 0.5 mm to 3 mm are further preferable. Alternatively, waveforms obtained by inverting, mixing, and rendering intermittent one-period waveforms as illustrated in FIG. 6A to FIG. 6I may also be used.

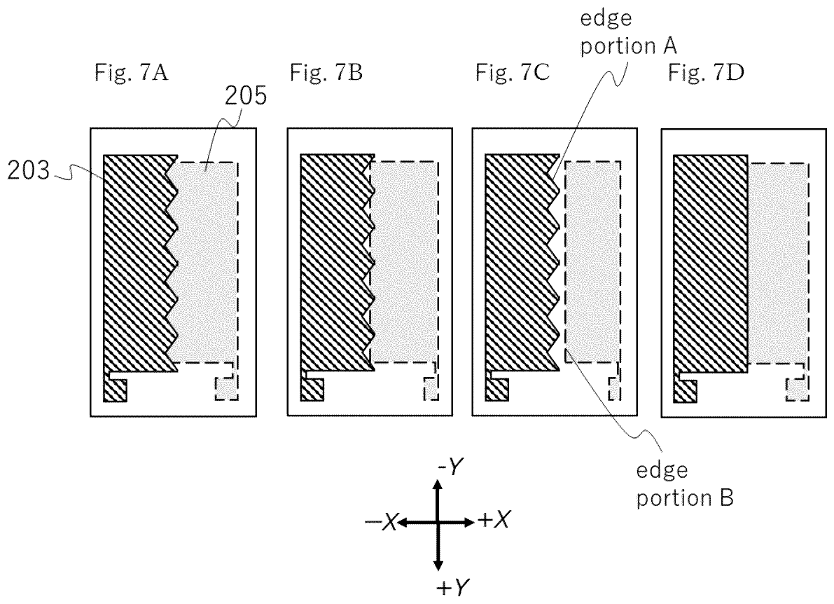

Using FIG. 7A to FIG. 7D, a description will be given of embodiments of the overlap.

FIG. 7A to FIG. 7D are illustrative views of the overlap between the first electrode 203 and the second electrode 205 of the plasma actuator.

The first electrode 203 and the second electrode 205 which are arranged to obliquely face each other are such that, when the plasma actuator is seen through from the first electrode (first surface) side, the edge portion of the first electrode is present in a portion where the second electrode is formed with the dielectric material being interposed therebetween. Specifically, the first electrode and the second electrode are provided so as to overlap each other with the dielectric material being interposed therebetween. In this case, it is preferable not to cause dielectric breakdown in a portion where the first electrode and the second electrode overlap each other with the dielectric material being interposed therebetween at the time of voltage application.

FIG. 7A illustrates an embodiment in which the first embodiment and the second embodiment overlap each other with the dielectric material being interposed therebetween (in the thickness direction). The embodiment is an example in which the first electrode and the second electrode overlap each other in the entire notched portion.

It is assumed that, when the plasma actuator is seen through from the first electrode 203 (first surface) side, the edge portion of the first electrode leading in the first direction (+X-direction) is the edge portion A, while the edge portion of the second electrode leading in the second direction (−X-direction) reverse to the first direction is the edge portion B. At this time, the edge portion B is located in the second direction (−X-direction) to be ahead of a most leading side of the notched portion of the edge portion A in the second direction.

Thus, the first electrode and the second electrode overlap each other with the dielectric material being interposed therebetween to allow the plasma and the induced flows to be stably generated.

In addition, since the first electrode and the second electrode are arranged to obliquely face each other via the dielectric material 201, the edge portion B is located ahead of the edge portion of the first electrode opposite to the edge portion A in the first direction (+X-direction). This can inhibit the induced flows from being generated from the edge portion of the first electrode opposite to the edge portion A.

FIG. 7B illustrates an embodiment in which the first electrode and the second electrode overlap each other with the dielectric material being interposed therebetween (in the thickness direction). This embodiment is an example in which the first electrode overlaps the second electrode in a portion of the notched portion (example in which the overlap is observed only in the notched portion).

It is assumed that, when the plasma actuator is seen through from the first electrode 203 (first surface) side, the edge portion of the first electrode leading in the first direction (+X-direction) is the edge portion A, while the edge portion of the second electrode leading in the second direction (−X-direction) reverse to the first direction is the edge portion B. At this time, the edge portion B is located between a most leading side of the notched portion of the edge portion A in the first direction and a most leading side of the notched portion of the edge portion A in the second direction. In other words, the edge portion B is located in the second direction (−X-direction) to be ahead of the most leading side of the notched portion of the edge portion A in the first direction, but is located in the first direction (+X-direction) to be ahead of the most leading side of the notched portion of the edge portion A in the second direction.

It is also preferable that the first electrode and the second electrode overlap each other only in the entire notched portion (only the depth 301-2 of the notched portion in FIG. 3B overlaps).

In other words, it is assumed that, when the plasma actuator is seen through from the first electrode 203 (first surface) side, the edge portion of the first electrode leading in the first direction (+X-direction) is the edge portion A, while the edge portion of the second electrode leading in the second direction (−X-direction) reverse to the first direction is the edge portion B. At this time, the edge portion B and the most leading side of the notched portion of the edge portion A in the second direction coincide with each other.

FIG. 7C illustrates an embodiment in which the first electrode and the second electrode do not overlap each other with the dielectric material being interposed therebetween (in the thickness direction).

It is assumed that, when the plasma actuator is seen through from the first electrode side, the edge portion of the first electrode leading in the first direction is the edge portion A, while the edge portion of the second electrode leading in the second direction (−X-direction) reverse to the first electrode is the edge portion B. At this time, the edge portion B is located in the first direction (+X-direction) to be ahead of the most leading side of the notched portion of the edge portion A in the first direction. In such an embodiment, no discharge occurs, and neither ozone nor induced flows are generated.

In the active oxygen supply device according to the present disclosure, when, e.g., an area of the surface to be treated of the object to be treated is large with respect to the opening portion, it is possible to perform the treatment while moving at least one of the active oxygen treatment device and the object to be treated. Relative moving speeds and movement directions of the active oxygen supply device and the object to be treated at that time may be set appropriately within a range in which the surface to be treated can be treated to an intended degree, and are not particularly limited. In addition, the number of times the object to be treated is treated may similarly be set appropriately within a range in which the surface to be treated can be treated to an intended degree.

### EXAMPLES

Using Examples and Comparative Examples, the following will describe the present disclosure in greater detail, but the embodiments of the present disclosure are not limited thereto.

### Example A

### 1. Production of Plasma Actuator A-1

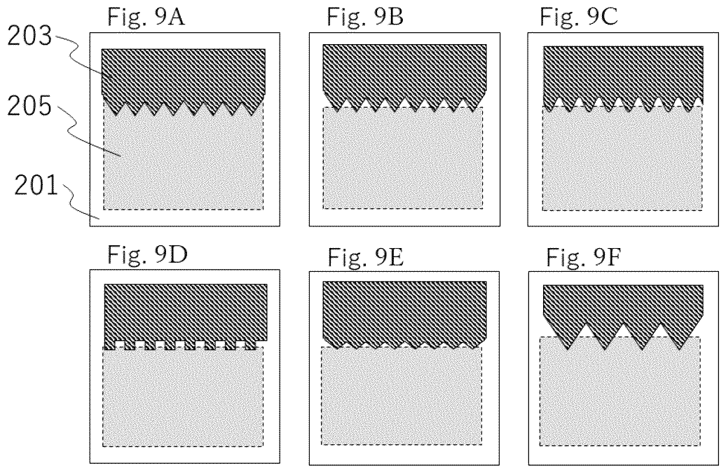

A plasma actuator A-1 illustrated in FIG. 9A was produced as follows. To a first surface of a glass plate (having a length of 5 mm, a width of 18 mm (in a paper surface depth direction in FIG. 1A), and a thickness of 150 μm) serving as the dielectric material, aluminum foil having a length of 2.5 mm, a width of 15 mm, and a thickness of 100 μm was bonded using an adhesive tape to form the first electrode. Meanwhile, to a second surface of the glass plate also, aluminum foil having a length of 3 mm, a width of 15 mm, and a thickness of 100 μm was bonded using an adhesive tape so as to obliquely face the aluminum foil bonded to the first surface and thereby form the second electrode. In addition, the second surface including the second electrode was covered with a polyimide tape. Note that an overlap amount 301 of the overlap between the first electrode and the second electrode was set to 2.2 mm. Moreover, the edge portion 204 of the first electrode was provided with a notch having isosceles triangular shapes. The shape of the notch was such that the depth 301-2 in the −X-direction was set to 2 mm, while a length 901 of a bottom side leading in the +X-direction was set to 2 mm. The angle Φ formed by the notch and illustrated in FIG. 6A was 53º.

### (Measurement of Ozone Amount in Induced Flows)

To calculate an amount of the ozone generated from the plasma actuator A-1, the plasma actuator A-1 was placed in an airtight container (not shown) having a volume of 1 liter. The airtight container was provided with an aperture portion that could be sealed with a rubber plug to allow a gas inside the container to be sucked with an injector from the aperture portion. Then, between the first electrode and the second electrode, an ac voltage (2.4 kVpp, 80 kHz) was applied and, after 60 seconds, 100 ml of the gas in the airtight container was collected. An ozone detector tube (Trade Name: 182SB, manufactured by Komei Rikagaku Kogyo Co., Ltd.) was caused to suck the collected gas to determine a measured ozone concentration (PPM) contained in the induced flows from the plasma actuator 103. Using the value of the measured ozone concentration, the amount of ozone generated per unit time was determined on the basis of the following expression:

$$Amount of ozone generated per unit time ( \frac{mg}{min} ) = Measured ozone concentration ( PPM ) * \frac{Ozone molecular weight 48}{22.4} * \frac{\frac{273}{273 + Room temperature ( ° C . )}}{10000} * \frac{Gas inside closed container ( L )}{Collected gas ( L )} = Measured ozone concentration ( PPM ) * 48 / 22.4 * 273 / ( 273 + 25 ) / 10000 * 0.1 / 1 \qquad [ Math . 1 ]$$

### (Measurement of Distances Reached by Induced Flows Containing Active Oxygen)

Using a decoloring reaction of methylene blue caused by active oxygen, which was disclosed in “Masanobu WAKASA et al., “Magnetic Field Effect on the Photocatalytic Reaction with TiO₂ Semiconductor Film”, Journal of The Society of Photographic Science and Technology of Japan, 69, 4, 271-275 (2006)”, distances reached by the induced flows containing the active oxygen from the edge portion 204 of the first electrode in the +X-direction were observed. The methylene blue is a crystalline powder with a blue luster, and the active oxygen (e.g., ·O₂⁻, ·OH) decomposes the methylene blue to cause a blue color to promptly disappear. Meanwhile, the ozone or the UV light does not cause the blue color to disappear in a short time. Accordingly, by observing distances over which the decoloring (blue color disappearance) is observed, it is possible to confirm the distances reached by the induced flows containing the active oxygen.

First, the methylene blue (manufactured by Kanto Chemical Co., Inc., special grade) and distilled water were mixed to prepare a 0.3% aqueous methylene blue solution. In the aqueous methylene blue solution, a filter paper sheet was immersed to absorb the methylene blue solution and be impregnated therewith.

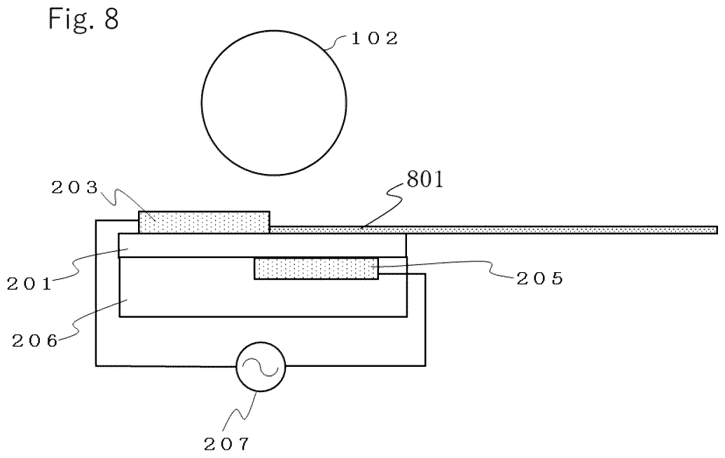

Then, on a side leading in a direction (first direction) in which the induced flows were blown from the first electrode including a space over the exposed surface 201-1 of the dielectric material 201 of the plasma actuator A-1, as illustrated in (FIG. 8), a filter paper sheet 801 impregnated with the methylene blue solution prepared above was placed. In addition, at a position where a shortest distance from the exposed surface 201-1 was 2 mm, as the UV light source 102, a UV lamp (cold cathode tube UV lamp, Trade Name: UW/9F89/9, manufactured by Stanley Electric Co., Ltd., having a cylindrical shape with a 9 mm diameter, peak wavelength=254 nm) was disposed.

Then, between the first electrode and the second electrode, an ac voltage (2.4 kVpp, 80 kHz) was applied, while the UV lamp was lighted, and a power source was cut off after 120 seconds to turn off the UV light. It was confirmed that, even when exposed to the ozone or the UV light for 120 seconds, the filter paper sheet impregnated with the methylene blue solution did not decolor. Accordingly, by observing a distance (hereinafter referred to also as the “decoloring distance”) from the edge portion 204 of the first electrode in the first direction over which decoloring of the methylene blue has occurred, it is possible to know the distances reached by the induced flows containing the active oxygen. Then, a distance to a leading end of a decolored portion most distant from the edge portion 204 was visually measured. Amounts of generated ozone and the decoloring distances are shown in Table 1.

### <Production of Plasma Actuator A-2 (for Comparison)>

A plasma actuator A-2 for comparison was produced similarly to the plasma actuator A-1 except that the edge portion 204 of the first electrode was provided with no notch in the plasma actuator A-1, as illustrated in FIG. 7D. Then, the ozone generation amount and the decoloring distance of the methylene blue were measured in the same manner as for the plasma actuator A-1. The results are shown in Table 1.

### <Production of Plasma Actuator A-3>

A plasma actuator A-3 was produced similarly to the plasma actuator A-1 except that the overlap amount 301 was set to 0.45 mm in the plasma actuator A-1 (FIG. 9B). Note that the overlap amount was adjusted such that, when the plasma actuator A-3 was seen through from the first electrode side, a total sum (corresponding to L¹ previously described) of lengths of portions (thick-line portions 110 in FIG. 10) of the edge portion 204 of the first electrode 203 which overlapped the second electrode 205 was equal to 15 mm, which was a length of the edge portion of the first electrode of the plasma actuator A-2.

As illustrated in Table 1, it was understood that the plasma actuators A-1 and A-3 each having the notched portion in the edge portion 204 according to the present disclosure had the distances reached by the active oxygen which were longer than that in the plasma actuator A-2.

In particular, each of the plasma actuator A-2 for comparison and the plasma actuator A-3 according to the present disclosure has the same discharge length in the edge portion 204 of the first electrode. When the plasma actuator A-2 and the plasma actuator A-3 were compared to each other, concentrations of the ozone caused in the induced flows were on the same level, but the distances reached by the induced flows containing the active oxygen were larger in the plasma actuator A-3. This may be conceivably because, as illustrated in FIG. 5, air flows 501 and 502 were generated in directions of electric lines of force directed from a portion of the edge portion of the first electrode which overlapped the second electrode to the second electrode, and these air flows joined together to increase a wind speed of the air flow 105-2 in the first direction, and consequently the active oxygen had reached far.

TABLE 1

| Plasma Actuator No. | A-1 | A-2 | A-3 |
|---|---|---|---|
| Ozone Concentration (μg/min) | 37 | 19 | 19 |
| Decoloring Distance (mm) | 8 | 1 | 5 |

### Example B-1

### 1. Preparation of Active Oxygen Supply Device

A plasma Actuator B-1 (FIG. 9A) was produced similarly to the plasma actuator A-1 described above. FIG. 9A illustrates a plan view obtained by viewing the plasma actuator from the first electrode side.

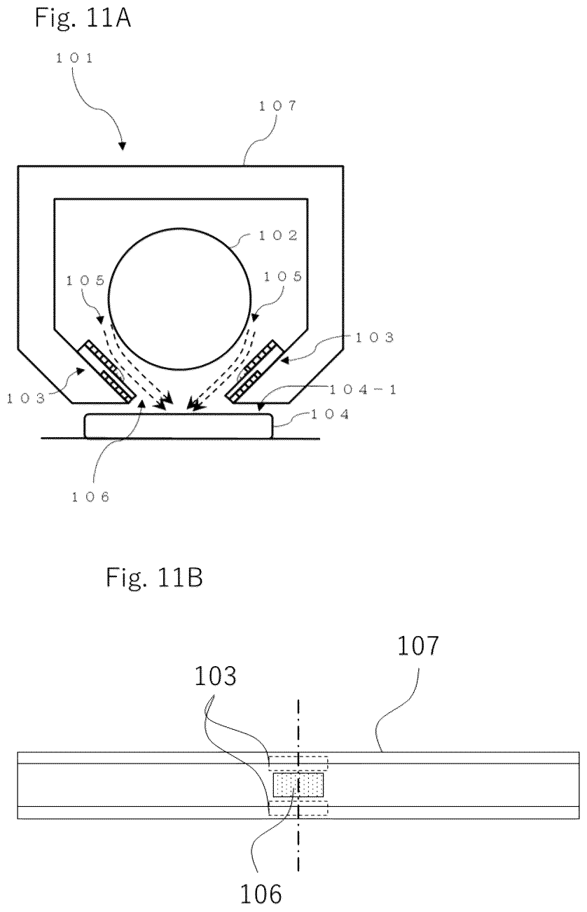

Then, as the housing 107 of the active oxygen supply device 101, a case made of an ABS resin, having a height of 25 mm, a width of 20 mm, a length of 170 mm, and a thickness of 2 mm, and having a cross-sectional shape (one-dot broken line in FIG. 11B) in a lateral direction, which was a substantially trapezoidal shape illustrated in FIG. 11A, was prepared. As illustrated in FIG. 11B which is a plan view obtained by viewing the case from an opening portion side, the case had the rectangular opening portion 106 having a width of 7 mm and a length of 15 mm laterally symmetrically with respect to a center (one-dot broken line in FIG. 11B) in the longitudinal direction. Then, to oblique side portions of the housing 107 in FIG. 11A, the two plasma actuators 103 produced in advance were fixed. An angle θ (having the same value as that of the PA incidence angle described above) formed at a point of intersection between the extension line 201-1-1 of the direction along the exposed portion 201-1 of the first surface of the dielectric material 201 of the plasma actuator 103 and the to-be-treated surface 104-1 of the to-be-treated object was 45°. In addition, at a position in the longitudinal direction of the housing at which the plasma actuator 103 is to be mounted, a center in the longitudinal direction of the housing and a center in the longitudinal direction (18 mm) of the plasma actuator were caused to coincide with each other, as illustrated in FIG. 11B.

Moreover, inside the housing, the UV lamp 102 (cold cathode tube UV lamp, Trade Name: UW/9F89/9, manufactured by Stanley Electric Co., Ltd., having a cylindrical shape with a 9 mm diameter, peak wavelength=254 nm) was disposed. The UV lamp 102 was disposed such that a distance (reference sign 403 in FIG. 4) between the UV lamp 102 and the exposed portion 201-1 of the first surface of the dielectric material 201 of the plasma actuator was 2 mm and that a distance (reference sign 401 in FIG. 4) between the UV light source and a surface of a flat plate which faced the UV light source when the flat plate was brought into contact with the opening portion 106 of the housing 107 was 3 mm. Thus, the active oxygen supply device (treatment device using the active oxygen) according to the present embodiment was produced.

At a position of the opening portion 106 serving as a feeding port for the active oxygen in this active oxygen supply device 101, an illuminometer (Trade Name: Spectral Radiometer USR-45D, manufactured by Ushio Inc.) was placed to measure the illuminance of the UV light. From an integration value of spectrum, 1370 μW/cm² was determined. At this time, the plasma actuator was not turned ON so as not be affected by blocking of the UV light by the ozone generated from the plasma actuator. Since the object to be treated was placed at, e.g., the position of the opening portion 106, the illuminance of the UV light measured under such conditions was regarded as the illuminance of the UV light at the surface of the object to be treated.

Subsequently, to calculate an amount of the ozone generated from the plasma actuator 103, the active oxygen supply device 101 was placed in an airtight container (not shown) having a volume of 1 liter. The airtight container was provided with an aperture portion that could be sealed with a rubber plug to allow a gas inside to be sucked with an injector from the aperture portion. Then, to the plasma actuator 103, a voltage of 2.4 kVpp having a sine waveform with a frequency of 80 kHz was applied without lighting the UV lamp and, after one minute, 100 ml of the gas inside the airtight container was collected. An ozone detector tube (Trade Name: 182SB, manufactured by Komei Rikagaku Kogyo Co., Ltd.) was caused to suck the collected gas to determine a measured ozone concentration (PPM) contained in the induced flows from the plasma actuator 103. Using the value of the measured ozone concentration, the amount of ozone generated per unit time was determined on the basis of the expression described above. As a result, the amount of ozone generated per unit time was 74 μg/minute.

Finally, the ozone generation amount when both of the plasma actuator 103 and the UV lamp 102 were operating was measured. An operation condition for the plasma actuator 103 was such that, when only the plasma actuator 103 was operated, 74 μg/minute of ozone was generated, as described above. Meanwhile, an operation condition for the UV lamp 102 was such that, when only the UV lamp 102 was operated, the illuminance was 1370 μW/cm², as described above. As a result, the ozone generation amount when both of the plasma actuator 103 and the UV lamp 102 were operating was 16 μg/minute. It can be considered that 58 μg/minute corresponding to a decrease from 74 μg/minute was the amount of ozone that had changed to the active oxygen.

### 2-1. Active Oxygen Detection Test (Methylene Blue Absorbance)

The active oxygen supply device produced in 1 described above was operated to confirm the presence or absence of the active oxygen in the induced flows flown out from the opening of the housing by using the decoloring reaction of the methylene blue. Specifically, the methylene blue (manufactured by Kanto Chemical Co., Inc., special grade) and distilled water were mixed to prepare a 0.01% aqueous methylene blue solution. 15 ml of the aqueous methylene blue solution was placed in a petri dish (AB4000 manufactured by Eiken Chem Co., Ltd. and having a cylindrical shape with a 88 mm diameter). Then, a liquid surface of the aqueous methylene blue solution in the petri dish was regarded as the to-be-treated surface 104-1 of the to-be-treated object, and the active oxygen supply device was placed on the petri dish such that a distance 405 in FIG. 4 was 6 mm.

Then, between the two electrodes of the plasma actuator, an ac voltage having a sine waveform with an amplitude of 2.4 kVpp and a frequency of 80 kHz was applied, while the UV lamp was lighted, and the induced flows flown out from the opening were supplied toward the liquid surface for 30 minutes. Note that the UV lamp was adjusted such that an illuminance at the position of the liquid surface was 1370 μW/cm² without turning ON the power source of the plasma actuator.

The aqueous methylene blue solution after the irradiation of the induced flows was moved to a cell, and changes in an amount of light absorbed in the methylene blue were measured using a spectrophotometer (V-570 manufactured by Jasco Corporation). Since the methylene blue exhibits strong absorption at a wavelength of 664 nm, a degree of decoloring of the methylene blue can be calculated from changes in absorbance at the wavelength. In the present test, first, only the distilled water was placed in a reference cell, and a 0.01% aqueous methylene blue solution before the irradiation of the induced flows was placed in a sample cell, and an absorbance thereof was measured to be 2.32 Abs. Meanwhile, the absorbance of the aqueous methylene blue solution after the irradiation of the induced flows was 0.05 Abs. Accordingly, a decrease rate of the absorbance was 97.8% ((2.32−0.05)/2.32×100).

### 2-2. Treatment (Sterilization) Test

Using the active oxygen supply device 101, an Escherichia coli sterilization test was carried out according to the following procedure. Note that all the instruments used in this sterilization test were sterilized with high-pressure steam using an autoclave. The present sterilization test was performed in a clean bench.

First, Escherichia coli (Trade Name “KWIK-STIK (Escherichia coli ATCC8739)”, manufactured by Microbiologics, Inc.) was placed in a conical flask containing 200 mL of distilled water in addition to a mixture of an LB medium (2 g of tryptone (Trade Name “Bacto Tryptone”, manufactured by Life Technologies Japan Ltd.), 1 g of yeast extract (Trade Name “Yeast Extract”, manufactured by Life Technologies Japan Ltd.), and 1 g of sodium chloride (Trade Name “Sodium Chloride special grade”, manufactured by Kishida Chemical Co., Ltd.). Subsequently, the conical flak was cultured with shaking at 80 rpm at 37° C. for 48 hours by using a shaking culture machine (TA-25R-3F manufactured by Takasaki Scientific Instruments Co., Ltd.) to obtain an Escherichia coli solution. The viable count of the obtained Escherichia coli solution was 9.2×109 (CFU/ml).

A sample No. 1 was produced by dropping 0.010 ml of the cultured bacterial solution only onto one surface of a qualitative filter paper sheet (Product Number: No. 5C, manufactured by Advantec Co., Ltd.) having a length of 3 cm and a width of 1 cm by using a micropipette. Sample No. 2 was similarly produced.

Then, Sample No. 1 was immersed for 1 hour in a test tube containing 10 ml of a buffer solution (Trade Name: Gibco PBS, manufactured by Thermo Fisher Scientific Inc.). Note that, to prevent the bacterial solution on the filter paper sheet from drying, the time from the dropping of the bacterial solution onto the filter paper sheet to the immersion into the buffer solution was set to 60 seconds.

Next, 1 ml of the buffer solution (hereinafter referred to also as the “1/1 solution”) after the immersion of Sample No. 1 therein was placed in a test tube containing 9 ml of the buffer solution to prepare a diluted solution (hereinafter referred to as the “1/10 diluted solution”). A 1/100 diluted solution, a 1/1000 diluted solution, and a 1/10000 diluted solution were prepared in the same manner, except that the dilution ratio with the buffer solution was changed.

Then, 0.050 ml was sampled from the 1/1 solution, and smeared on a stamp medium (PETAN CHECK 25 PT1025, manufactured by Eiken Chemical Co., Ltd.). This operation was repeated to prepare two stamp media smeared with the 1/1 solution. The two stamp media were placed in a thermostat (Trade Name: IS600, manufactured by Yamato Scientific Co., Ltd.) and cultured at a temperature of 37° C. for 24 hours. The numbers of colonies generated in the two stamp media were counted, and an average value thereof was calculated.

For each of the 1/10 diluted solution, the 1/100 diluted solution, the 1/1000 diluted solution, and the 1/10000 diluted solution also, two smeared stamp media were produced and cultured in the same manner as described above. Then, the numbers of colonies generated in the individual stamp media were counted, and an average value thereof was calculated. Table 2-1 shows the results.

TABLE 2-1

| | Sample No. 1 (Blank) |
|---|---|
| 1/1 Solution | >100 |
| 1/10 Solution | >100 |
| 1/100 Solution | 54 |
| 1/1000 Solution | 4 |
| 1/10000 Solution | 0 |

From the results shown in Table 2-1 above, it was found that the number of the colonies when the 1/100 diluted solution was cultured was 54, and therefore the number of bacteria present in 0.050 ml of the 1/1 solution related to Sample No. 1 was 54×10²=5400 (CFU).

Next, the following operation was performed with respect to Sample No. 2.

In a center of a plastic flat plate having a length of 30 cm, a width of 30 cm, and a thickness of 5 mm, a depressed portion having a length of 3.5 cm, a width of 1.5 cm, and a depth of 4.4 mm was provided. A filter paper sheet having a length of 3.5 cm and a width of 1.5 cm was laid in the depressed portion. On the filter paper sheet, Sample No. 2 was placed such that the bacterial-solution-dropped lower surface thereof faced the filter paper sheet laid on a bottom portion of the depressed portion. Then, on an upper surface of the plastic plate, the active oxygen supply device was placed such that a center of the opening thereof in the longitudinal direction coincided with a center of the depressed portion in the longitudinal direction and that a center of the opening thereof in the width direction coincided with a center of the depressed portion in the lateral direction. At this time, the distance 405 (distance from a leading end of the plasma actuator on the opening side to a surface of the filter paper sheet facing the UV lamp) illustrated in FIG. 4 was set to 6 mm.

Since the depth of the depressed portion was 4.4 mm and the thickness of the filter paper sheet was about 0.2 mm, the surface of each of the samples to which the bacterial solution had adhered did not come into direct contact with the opening of the active oxygen supply device. Then, between the two electrodes of the plasma actuator, an ac voltage having a sine waveform with an amplitude of 2.4 kVpp and a frequency of 80 kHz was applied, while the UV lamp was lighted to supply the induced flows toward the filter paper sheet. A supply time (treatment time) was set to 2 seconds. Note that the UV lamp was adjusted such that the illuminance measured on the surface of the filter paper sheet which faced the UV lamp was 1370 μW/cm².

In a treatment process using the active oxygen supply device, to prevent the filter paper sheet to which the bacterial solution was dropped from drying, the time from the dropping of the bacterial solution onto the filter paper sheet to the immersion into the buffer solution was set to 60 seconds.

Sample No. 2 after the treatment was immersed together with the filter paper sheet laid on the bottom portion of the depressed portion for 1 hour in a test tube containing 10 ml of a buffer solution (Trade Name: Gibco PBS, available from Thermo Fisher Scientific Inc.). Then, 1 ml of the buffer solution after the immersion (hereinafter referred to also as the “1/1 solution”) was placed in a test tube containing 9 ml of the buffer solution to prepare a diluted solution (1/10 diluted solution). A 1/100 diluted solution, a 1/1000 diluted solution, and a 1/10000 diluted solution were prepared in the same manner, except that the dilution ratio with the buffer solution was changed.

Then, 0.050 ml was sampled from the 1/1 solution, and smeared on a stamp medium (Trade Name: PETAN CHECK 25 PT1025, manufactured by Eiken Chemical Co., Ltd.). This operation was repeated to prepare two stamp media smeared with the 1/1 solution. A total of the two stamp media were placed in a thermostat (Trade Name: IS600, manufactured by Yamato Scientific Co., Ltd.) and cultured at a temperature of 37° C. for 24 hours. The numbers of colonies generated in the individual stamp media related to the 1/1 solution were counted, and an average value thereof was calculated. For each of the 1/10 diluted solution, the 1/100 diluted solution, the 1/1000 diluted solution, and the 1/10000 diluted solution also, two smeared stamp media were produced and cultured in the same manner as described above. Then, the numbers of colonies generated in the individual stamp media related to each of the diluted solutions were counted, and an average value thereof was calculated. Table 2-2 shows the results.

TABLE 2-2

| | Sample No. 2 |
|---|---|
| 1/1 Solution | 0 |
| 1/10 Solution | 0 |
| 1/100 Solution | 0 |
| 1/1000 Solution | 0 |
| 1/10000 Solution | 0 |

As shown in Table 2-1, the number of bacteria in 0.050 ml of the 1/1 solution related to Sample No. 1 that was not treated with the active oxygen supply device was 5400 (CFU) while, the number of bacteria in 0.050 ml of the 1/1 solution related to Sample No. 2 after the treatment was 0 (CFU). From this, it was found that, by the 2 second treatment using the active oxygen supply device according to the present embodiment, 100.00% (5400−0/5400×100) sterilization was achieved.

### Example B-2

A plasma actuator B-2 was produced similarly to the plasma actuator B-1 in Example 1 except that the overlap amount 301 in the plasma actuator B-1 was changed to 0.45 mm (see FIG. 9B). The overlap amount was adjusted such that, when the plasma actuator a was seen through from the first electrode side, a total sum (L¹) of the lengths of the portions (thick-line portions 110 in FIG. 10) of the edge portion 204 of the first electrode 203 which overlapped the second electrode 205 was 15 mm. An active oxygen supply device was produced in the same manner as in Example B-1 except that the plasma actuator B-2 was used. Then, the active oxygen supply device was subjected to the measurement of the ozone generation amount, the active oxygen detection test, and the sterilization test.

### Example 3

The notch of the edge portion 204 of the first electrode was provided with a sine wave shape having an amplitude of 1 mm and a wavelength of 2 mm, and the amount of the overlap with the second electrode was adjusted such that L¹ was 15 mm. Otherwise, a plasma actuator B-3 was produced similarly to the plasma actuator B-1 (see FIG. 9C). An active oxygen supply device was produced in the same manner as in Example B-1 except that the plasma actuator B-3 was used. Then, the active oxygen supply device was subjected to the measurement of the ozone generation amount, the active oxygen detection test, and the sterilization test.

### Example 4

The notch of the edge portion 204 of the first electrode was provided with a rectangular wave shape having an amplitude of 1 mm and a wavelength of 2 mm, and the amount of the overlap with the second electrode was adjusted such that L¹ was 15 mm. Otherwise, a plasma actuator B-4 was produced similarly to the plasma actuator B-1 (see FIG. 9D). An active oxygen supply device was produced in the same manner as in Example B-1 except that the plasma actuator B-4 was used. Then, the active oxygen supply device was subjected to the measurement of the ozone generation amount, the active oxygen detection test, and the sterilization test.

### Example 5

An amplitude of the notch of the edge portion 204 of the first electrode of the plasma actuator B-1 was reduced to 1/2 (0.5 mm), and the overlap amount was adjusted such that L¹ was 15 mm. Otherwise, a plasma actuator B-5 was produced similarly to the plasma actuator B-1 (see FIG. 9E). Then, the plasma actuator B-5 was subjected to the measurement of the ozone generation amount, the active oxygen detection test, and the sterilization test.

### Example 6

A wavelength and the amplitude of the notch of the edge portion 204 of the first electrode of the plasma actuator B-1 were doubled (an amplitude of 2 mm, a wavelength of 4 mm), and the overlap amount was adjusted such that L¹ was 15 mm. Otherwise, a plasma actuator B-6 was produced similarly to the plasma actuator B-1 (see FIG. 9F). Then, the plasma actuator B-6 was subjected to the measurement of the ozone generation amount, the active oxygen detection test, and the sterilization test.

Table 3 shows structural outlines of the plasma actuators B-1 to B-6.

TABLE 3

| Plasma Actuator | Discharge Length L¹ | |
|---|---|---|
| No. | (mm) | L¹/L² |
| B-1 | 34 | 2.3 |
| B-2 | 15 | 1.0 |
| B-3 | 15 | 1.0 |
| B-4 | 15 | 1.0 |
| B-5 | 15 | 1.0 |
| B-6 | 15 | 1.0 |

### <Comparative Examples B-1 to B-2>

In Comparative Examples B-1 to B-2, the same conditions as used in Example B-1 were used except that the following respective configurations were provided.

Comparative Example 1: No voltage was applied to the plasma actuator, and no UV light was applied thereto.

Comparative Example 2: No voltage was applied to the plasma actuator, but UV light was applied thereto.

Table 4 shows results of evaluating the active oxygen supply devices according to Examples 1 to 6 and Comparative Examples 1 to 2. Note that, as described above, each of the ozone concentrations was an ozone concentration when the UV lamp was not lighted, and only the plasma actuator was operated. Meanwhile, each of the UV light illuminances has a value when the illuminance was measured at the exposed surface of the dielectric material of the plasma actuator which faced the UV lamp in a state where the plasma actuator was not operated.

TABLE 4

| | | | | Decrease Rate of | | |
|---|---|---|---|---|---|---|
| | | | | Absorbance of | Sterilization Test | |
| | | Ozone | UV Lamp | Methylene Blue | Number of Bacteria in 1/1 | Sterilization |
| | | Concentration | Illuminance | Solution | Solution After Treatment | Rate |
| | | (μg/min) | (μW/cm²) | (%) | (CFU)/0.050 ml | (%) |
| Examples | 1 | 74 | 1370 | 97.8 | 0 | 100.00 |
| | 2 | 39 | 1370 | 93.0 | 21 | 99.60 |
| | 3 | 39 | 1370 | 91.0 | 32 | 99.40 |
| | 4 | 39 | 1370 | 84.0 | 102 | 98.10 |
| | 5 | 39 | 1370 | 89.0 | 77 | 98.60 |
| | 6 | 39 | 1370 | 86.0 | 89 | 98.40 |
| Comparative | 1 | 74 | 0 | 43.0 | 4900 | 9.30 |
| Examples | 2 | 0 | 1370 | 3.0 | 2100 | 61.10 |

In Comparative Example 1, a certain degree of sterilization effect by ozone was observed, but was not as high as those in Examples 1 to 6. In Comparative Example 2, a certain degree of sterilization effect by UV light was observed, but was far lower than those in Examples 1 to 6.

The present disclosure is not limited to the above embodiments, and various changes and modifications can be made therein without departing from the spirit and scope of the present disclosure. Therefore, to set out the scope of the present disclosure, the following claims are hereby appended.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

What is claimed is:

1. An active oxygen supply device comprising, inside a housing having at least one opening portion:
a plasma actuator; and
an ozone decomposing device, wherein
the plasma actuator comprises a first electrode, a dielectric material, and a second electrode which are stacked in this order,
the first electrode is an exposed electrode provided on a first surface, which is one surface of the dielectric material,
when a voltage is applied between the first electrode and the second electrode, the plasma actuator generates a dielectric barrier discharge directed from the first electrode to the second electrode and cause an induced flow containing ozone to be blown out from the first electrode in a first direction, which is one direction along the surface of the dielectric material,
the ozone decomposing device decomposes the ozone contained in the induced flow to generate active oxygen in the induced flow, and the induced flow results in an induced flow containing the active oxygen,
the plasma actuator and the ozone decomposing device are arranged such that the induced flow containing the active oxygen flows out from the opening portion to the outside of the housing,
when the plasma actuator is seen through from the first electrode side, at least one portion of an edge portion of the first electrode leading in the first direction overlaps the second electrode,
when the plasma actuator is viewed from the first electrode side, the edge portion of the first electrode has a notched portion,
the notched portion has a shape which increases a flow rate of the induced flow compared to that in a case where the edge portion of the first electrode does not have the notched portion, and
the ozone decomposing device is at least one device selected from the group consisting of:
a UV light source that irradiates the induced flow containing the ozone with UV light to generate the active oxygen in the induced flow; and
a heating device that heats the induced flow containing the ozone to generate the active oxygen in the induced flow.

2. The active oxygen supply device according to claim 1, wherein,
when a cross section of the plasma actuator along a thickness direction thereof is viewed,
the first electrode and the second electrode are arranged to obliquely face each other via the dielectric material in the thickness direction of the plasma actuator,
the first electrode is provided so as to cover a portion of the first surface of the dielectric material, and
the first surface has an exposed portion uncovered with the first electrode,
when the plasma actuator is seen through from the first electrode side, at least one portion of the exposed portion and the second electrode have overlap therebetween, and
in the cross section along the thickness direction, the induced flow is blown out from the edge portion of the first electrode leading in the first direction along the exposed portion of the dielectric material overlapping the second electrode.

3. The active oxygen supply device according to claim 1, wherein, when the plasma actuator is viewed from the first electrode side, the shape of the notched portion is a shape in which a width of a notch increases in the first direction.

4. The active oxygen supply device according to claim 1, wherein the notched portion has a triangular shape, a substantially triangular shape, a sawtooth shape, an arc shape, an ellipsoidal arc shape, a substantially arc shape, a sine wave shape, a trapezoidal shape, a substantially trapezoidal shape, a rectangular shape, or a substantially rectangular shape.

5. The active oxygen supply device according to claim 1, wherein the notched portion is provided at each of a plurality of positions in the edge portion.

6. The active oxygen supply device according to claim 5, wherein the notched portion is regularly present in the edge portion.

7. The active oxygen supply device according to claim 5, wherein the notched portion is periodically present in the edge portion.

8. The active oxygen supply device according to claim 1, wherein, when a total sum of lengths of portions overlapping the second electrode of the edge portion of the first electrode leading in the first direction having the notched portion is L¹ and when, assuming that the edge portion of the first electrode leading in the first direction does not have the notched portion, a length of a portion of the edge portion which overlaps the second electrode in a direction along the surface of the dielectric material is L², L¹/L² is 1.0 to 5.0.

9. The active oxygen supply device according to claim 1, wherein, assuming that the plasma actuator is seen through from the first electrode side, when the edge portion of the first electrode leading in the first direction is an edge portion A and when an edge portion of the second electrode leading in a second direction reverse to the first direction is an edge portion B, the edge portion B is located in the second direction to be ahead of a most leading side of the notched portion of the edge portion A in the second direction.

10. The active oxygen supply device according to claim 1, wherein, assuming that the plasma actuator is seen through from the first electrode side, when the edge portion of the first electrode leading in the first direction is an edge portion A and when an edge portion of the second electrode leading in a second direction reverse to the first direction is an edge portion B, the edge portion B is located between a most leading side of the notched portion of the edge portion A in the first direction and a most leading side of the notched portion of the edge portion A in the second direction.

11. The active oxygen supply device according to claim 1, wherein, assuming that the plasma actuator is seen through from the first electrode side, when the edge portion of the first electrode leading in the first direction is an edge portion A and when an edge portion of the second electrode leading in a second direction reverse to the first direction is an edge portion B, the edge portion B coincides with a most leading side of the notched portion of the edge portion A in the second direction.

12. The active oxygen supply device according to claim 1, wherein the ozone decomposing device further includes a humidifying device that humidifies the induced flow containing the ozone to generate the active oxygen in the induced flow.

13. A device for performing treatment with active oxygen, the device treating a surface of an object to be treated with the active oxygen and comprising, inside a housing having at least one opening portion:
a plasma actuator; and
an ozone decomposing device, wherein
the plasma actuator comprises a first electrode, a dielectric material, and a second electrode which are stacked in this order,
the first electrode is an exposed electrode provided on a first surface, which is one surface of the dielectric material,
when a voltage is applied between the first electrode and the second electrode, the plasma actuator generates a dielectric barrier discharge directed from the first electrode to the second electrode and cause an induced flow containing ozone to be blown out from the first electrode in a first direction, which is one direction along the surface of the dielectric material,
the ozone decomposing device decomposes the ozone contained in the induced flow to generate active oxygen in the induced flow, and the induced flow results in an induced flow containing the active oxygen,
the plasma actuator and the ozone decomposing device are arranged such that the induced flow containing the active oxygen flows out from the opening portion to the outside of the housing,
when the plasma actuator is seen through from the first electrode side, at least one portion of an edge portion of the first electrode leading in the first direction overlaps the second electrode,
when the plasma actuator is viewed from the first electrode side, the edge portion of the first electrode has a notched portion,
the notched portion has a shape which increases a flow rate of the induced flow compared to that in a case where the edge portion of the first electrode does not have the notched portion, and
the ozone decomposing device is at least one device selected from the group consisting of:
a UV light source that irradiates the induced flow containing the ozone with UV light to generate the active oxygen in the induced flow; and
a heating device that heats the induced flow containing the ozone to generate the active oxygen in the induced flow.

14. A treatment method for treating a surface of an object to be treated with active oxygen, the treatment method comprising:
a step of preparing a device for performing the treatment with the active oxygen, wherein
the device for performing the treatment with the active oxygen comprises, inside a housing having at least one opening portion:
a plasma actuator; and
an ozone decomposing device,
the plasma actuator comprises a first electrode, a dielectric material, and a second electrode which are stacked in this order,
the first electrode is an exposed electrode provided on a first surface, which is one surface of the dielectric material,
when a voltage is applied between the first electrode and the second electrode, the plasma actuator generates a dielectric barrier discharge directed from the first electrode to the second electrode and cause an induced flow containing ozone to be blown out from the first electrode in a first direction, which is one direction along the surface of the dielectric material,
the ozone decomposing device decomposes the ozone contained in the induced flow to generate active oxygen in the induced flow, and the induced flow results in an induced flow containing the active oxygen,
the plasma actuator and the ozone decomposing device are arranged such that the induced flow containing the active oxygen flows out from the opening portion to the outside of the housing,
when the plasma actuator is seen through from the first electrode side, at least one portion of an edge portion of the first electrode leading in the first direction overlaps the second electrode,
when the plasma actuator is viewed from the first electrode side, the edge portion of the first electrode has a notched portion,
the notched portion has a shape which increases a flow rate of the induced flow compared to that in a case where the edge portion of the first electrode does not have the notched portion, and
the ozone decomposing device is at least one device selected from the group consisting of:
a UV light source that irradiates the induced flow containing the ozone with UV light to generate the active oxygen in the induced flow; and
a heating device that heats the induced flow containing the ozone to generate the active oxygen in the induced flow,
the treatment method further comprising:
a step of disposing the prepared device for performing the treatment with the active oxygen and the object to be treated at relative positions where a surface of the object to be treated is exposed to the induced flow when the induced flow is caused to flow out from the opening portion; and
a step of causing the induced flow to flow out from the opening portion to treat the surface of the object to be treated with the active oxygen.
